(19) United States
(12) Patent Application Publication
(10) Pub. No.: US 2015/0164069 A1
(43) Pub. Date: Jun. 18, 2015

(54) INSECTICIDAL LIPID AGENTS ISOLATED FROM ENTOMOPATHOGENIC FUNGI AND USES THEREOF

(71) Applicants: Stephen Reynold Ford, Auckland (NZ); Travis Robert Glare, Lincoln (NZ); Cody Calder, Auckland (NZ); Roelof Van Ginkel, Nelson (NZ)

(72) Inventors: Stephen Reynold Ford, Auckland (NZ); Travis Robert Glare, Lincoln (NZ); Cody Calder, Auckland (NZ); Roelof Van Ginkel, Nelson (NZ)

(73) Assignee: BIOTELLIGA HOLDINGS LIMITED, Auckland (NZ)

(21) Appl. No.: 14/342,316

(22) PCT Filed: Aug. 31, 2012
(86) PCT No.: PCT/IB2012/054483

§ 371 Date: Feb. 19, 2015

Related U.S. Application Data
(60) Provisional application No. 61/530,168, filed on Sep. 1, 2011.

(51) Int. Cl. A01N 37/44; A01N 63/04; C12P 7/64; C07C 229/22

(52) U.S. Cl. CPC A01N 37/44; C07C 229/22; A01N 63/04; C12P 7/6436

(57) ABSTRACT

The present invention provides insecticidal lipids together with compositions comprising such lipids and lipid fractions and methods of preparing same. Methods for the biological control of insects, such as phytopathogenic insects, using the lipids or compositions comprising said lipids optionally together with one or more insecticidal or entomopathogenic agents including entomopathogenic fungi, are also provided.

### FIELD OF THE INVENTION

This invention relates generally to the field of biology, more particularly certain embodiments concerning lipids including lipids prepared from filamentous fungi, compositions comprising said lipids, and the use of such lipids and compositions as biological control agents. Methods for the control of insects, including phytopathogenic insects, using the lipids and compositions comprising the lipids are also provided.

### BACKGROUND OF THE INVENTION

The ability to control insects and insect populations is of significant importance to human and animal health, agriculture, and a wide range of economic activities. For example, insects are vectors for a number of important human diseases: mosquitoes are vectors for malaria, West Nile disease, and Dengue fever, ticks are vectors for rickettsial disease such as typhus, African tick bite fever, and Lyme disease, while fleas are the vector for plague. Similarly, plant disease or loss caused by insect pests and pathogens (collectively “phytopathogens”), is a significant economic cost to plant-based agriculture and industries. Losses may arise through spoilage of produce both pre and post harvest, loss of plants themselves, or through reduction in growth and production abilities.

Traditionally, large scale control of insects, such as plant pests and pathogens, has been pursued through the application of chemical insecticides through physical methods (e.g., trapping, picking, barriers) may also be employed. The use of chemicals is subject to a number of disadvantages. Insects, such as plant pests and pathogens, can and have developed tolerance to chemicals to over time, producing resistant populations. Indeed, resistance to pesticides is the greatest challenge to the viability of plant-based agriculture and industries such as the horticultural industry.

The problem is particularly illustrated with reference to a number of economically important phytopathogenic insects. Populations of western flower thrips worldwide are reported to be resistant to most groups of pesticides including the following examples; acephate, abamectin, chlorpyrifos, endosulfan, methomyl, methiocarb, omethoate, pyrazophos and tau-fluvalinate. Populations of onion thrips in New Zealand have developed resistance to deltamethrin, and local populations have been reported to be resistance to diazinon and dichlorvos. Onion thrips in the United States have been reported to be resistant to many pesticides (Grossman, 1994). Greenhouse whitefly has reportedly developed resistance to organochlorine, organophosphate, carbamate and pyrethroid insecticides (e.g. Georghiou 1981, Anis & Brennan 1982, Elhag & Horn 1983, Wardlow 1985, and Hommes 1986). Resistance has also been reported in newer insecticides, buprofezin and teflubenzuron (Gorman et al. 2000).

Chemical residues may also pose environmental hazards, and raise health concerns. The revival of interest in biological control such as microbial insecticides over the last 20 years has come directly, from public pressure in response to concerns about chemical toxicities. Biological control presents an alternative means of controlling plant pathogens which is potentially more effective and specific than current methods, as well as reducing dependence on chemicals. Such biological control methods are perceived as a “natural” alternative to chemical insecticides with the advantage of greater public acceptance, reduced environmental contamination, and increased sustainability.

Mechanisms of biological control are diverse. One mechanism which has been demonstrated to be effective is the use of antagonistic microorganisms such as bacteria to control phytopathogenic insects. For example, the large scale production of Bacillus thuringiensis enabled the use of this bacterio-insecticide to control painted apple moth in Auckland, New Zealand.

There are, however, few examples of the successful application of biological control agents (BCAs), and to date BCAs have not met with significant grower acceptance and may have been perceived to be uneconomic.

There is thus a need for agents and methods for effectively controlling insects, including phytopathogenic insects, particularly agents that act faster, have increased efficacy in controlling insects, require less frequent or less intensive application, have lower cost, or have lower resulting toxicity than the currently-available insecticides.

It is therefore an object of the present invention to go some way to meeting this need, to provide one or more agents and methods useful in the control of insects and insect populations, including phytopathogenic insects, or at least to provide the public with a useful choice.

### SUMMARY OF THE INVENTION

The present invention provides insecticidal lipids and methods for preparing them. The dissimilarity of these lipids to known insecticides indicates they comprise a new class of insecticidal agents.

Accordingly, in a first aspect the invention relates to an isolated, purified or substantially pure lipid of formula

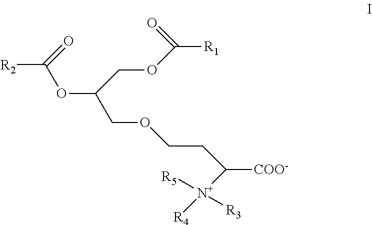

wherein R₁ and R₂, which can be the same or different, are each the aliphatic portion of a fatty acid, and wherein R3, R4 and R5 are each independently selected from the group comprising or consisting of H, hydroxyl, carboxyl, amide, unsubstituted or substituted alkyl, unsubstituted or substituted alkenyl, unsubstituted or substituted alkynyl, and unsubstituted or substituted aryl moieties, for use in the control of one or more insects.

In one embodiment, R₁ and R₂ are independently selected from the group comprising the aliphatic portions of C16 saturated fatty acids, C18 saturated fatty acids, C18 mono-unsaturated fatty acids, and C18 di-unsaturated fatty acids.

In one embodiment, R₁ or R₂ or both R₁ and R₂ is the aliphatic portion of a C16 saturated fatty acid.

In one embodiment, R₁ or R₂ or both R₁ and R₂ is the aliphatic portion of a C18 saturated fatty acid.

In one embodiment, R₁ or R₂ or both R₁ and R₂ is the aliphatic portion of a C18 mono-unsaturated fatty acid.

In one embodiment, R₁ or R₂ or both R₁ and R₂ is the aliphatic portion of a C18 di-unsaturated fatty acid.

In one embodiment, R₁ or R₂ or both R₁ and R₂ is the aliphatic portion of a C18 tri-unsaturated fatty acid.

In one embodiment, R₁ is the aliphatic portion of a C16 saturated fatty acid. In a further embodiment, R₁ is the aliphatic portion of a C16 saturated fatty acid and R₂ is the aliphatic portion of a C18 mono-unsaturated fatty acid. In a still further embodiment, R₁ is the aliphatic portion of a C16 saturated fatty acid and R₂ is the aliphatic portion of a C18 di-unsaturated fatty acid. In a still further embodiment, R₁ is the aliphatic portion of a C16 saturated fatty acid and R₂ is the aliphatic portion of a C18 tri-unsaturated fatty acid.

In one embodiment, R₂ is the aliphatic portion of a C16 saturated fatty acid. In a further embodiment, R₂ is the aliphatic portion of a C16 saturated fatty acid and R₁ is the aliphatic portion of a C18 mono-unsaturated fatty acid. In a still further embodiment, R₂ is the aliphatic portion of a C16 saturated fatty acid and R₁ is the aliphatic portion of a C18 di-unsaturated fatty acid. In a still further embodiment, R₂ is the aliphatic portion of a C16 saturated fatty acid and R₁ is the aliphatic portion of a C18 tri-unsaturated fatty acid.

In one embodiment, R₁ is the aliphatic portion of a C18 saturated fatty acid. In a further embodiment, R₁ is the aliphatic portion of a C18 saturated fatty acid and R₂ is the aliphatic portion of a C18 mono-unsaturated fatty acid. In a still further embodiment, R₁ is the aliphatic portion of a C18 saturated fatty acid and R₂ is the aliphatic portion of a C18 di-unsaturated fatty acid. In a still further embodiment, R₁ is the aliphatic portion of a C18 saturated fatty acid and R₂ is the aliphatic portion of a C18 tri-unsaturated fatty acid.

In one embodiment, R₂ is the aliphatic portion of a C18 saturated fatty acid. In a further embodiment, R₂ is the aliphatic portion of a C18 saturated fatty acid and R₁ is the aliphatic portion of a C18 mono-unsaturated fatty acid. In a still further embodiment, R₂ is the aliphatic portion of a C18 saturated fatty acid and R₁ is the aliphatic portion of a C18 di-unsaturated fatty acid. In a still further embodiment, R₂ is the aliphatic portion of a C18 saturated fatty acid and R₁ is the aliphatic portion of a C18 tri-unsaturated fatty acid.

In one embodiment, R₁ is the aliphatic portion of a C18 mono-unsaturated fatty acid. In a further embodiment, R₁ is the aliphatic portion of a C18 mono-unsaturated fatty acid and R₂ is the aliphatic portion of a C18 mono-unsaturated fatty acid. In a still further embodiment, R₁ is the aliphatic portion of a C18 mono-unsaturated fatty acid and R₂ is the aliphatic portion of a C18 di-unsaturated fatty acid. In a still further embodiment, R₁ is the aliphatic portion of a C18 mono-unsaturated fatty acid and R₂ is the aliphatic portion of a C18 tri-unsaturated fatty acid.

In one embodiment; R₂ is the aliphatic portion of a C18 mono-unsaturated fatty acid. In a further embodiment, R₂ is the aliphatic portion of a C18 mono-unsaturated fatty acid and R₁ is the aliphatic portion of a C18 mono-unsaturated fatty acid. In a still further embodiment, R₂ is the aliphatic portion of a C18 mono-unsaturated fatty acid and R₁ is the aliphatic portion of a C18 di-unsaturated fatty acid. In a still further embodiment, R₂ is the aliphatic portion of a C18 mono-unsaturated fatty acid and R₁ is the aliphatic portion of a C18 tri-unsaturated fatty acid.

In one embodiment, R₁ is the aliphatic portion of a C18 di-unsaturated fatty acid. In a further embodiment, R₁ is the aliphatic portion of a C18 di-unsaturated fatty acid and R₂ is the aliphatic portion of a C18 di-unsaturated fatty acid. In a still further embodiment, R₂ is the aliphatic portion of a C18 di-unsaturated fatty acid. In a still further embodiment, R₁ is the aliphatic portion of a C18 di-unsaturated fatty acid and R₂ is the aliphatic portion of a C18 tri-unsaturated fatty acid.

In one embodiment, R₁ is the aliphatic portion of a C18 tri-unsaturated fatty acid. In a further embodiment, R₁ is the aliphatic portion of a C18 tri-unsaturated fatty acid and R₂ is the aliphatic portion of a C18 tri-unsaturated fatty acid. In a still further embodiment, R₁ is the aliphatic portion of a C18 tri-unsaturated fatty acid and R₂ is the aliphatic portion of a C18 di unsaturated fatty acid.

In one embodiment, R₃, R₄, R₅ are each H or methyl. In another embodiment, R₃, R₄, R₅ are each methyl.

In one embodiment, the lipid is a betaine lipid.

In one embodiment the lipid is 1,2-diacylglyceryl-3-O-4′-(N,N,N-trimethyl)-homoserine (diacylglyceryl-N,N,N-trimethylhomoserine, DGTS). In another embodiment, the lipid is 1,2-diacylglyceryl-3-O-2′-(hydroxymethyl)-(N,N,N-trimethyl)-β-alanine, or 1,2-diacylglyceryl-3-O-carboxy-(hydroxymethyl)-choline.

The invention provides compositions and formulations that comprise one or more of the lipids disclosed herein, together with at least one pharmaceutically- or agriculturally-acceptable carrier, including compositions and formulations comprising one or more lipids of the invention and one or more fungi. Such compositions may be a cell extract, cell suspension, cell homogenate, cell lysate, cell supernatant, cell filtrate, or cell pellet of a cell that produce such lipids. In certain exemplary embodiments, the composition is enriched in one or more lipids of the invention, for example by purification or addition. In other exemplary embodiments, the composition comprises one or more added lipids of the invention. For example, in one embodiment the composition is a cell extract, cell suspension, cell homogenate, cell lysate, cell supernatant, cell filtrate, or cell pellet as described above to which has been added one or more lipids of the invention.

Thus, in a second aspect the invention relates to a composition comprising one or more lipids of the invention, together with one or more carriers.

In a further aspect, the invention provides a method of preparing a lipid having insecticidal activity against an insect, such as for example a sucking insect, or a coleopteran, dipteran, or lepidopteran insect. The method generally involves isolating one or more of the lipids described herein from a suitable culture of cells, such as a culture of one or more fungi of the phylum Ascomycota. In certain embodiments the cells are or have been grown under conditions capable of supporting mycelial growth. Such lipids may be isolated from the cell culture or supernatant or from spore suspensions derived from the cell culture and used in the native form, or may be otherwise purified or concentrated as appropriate for the particular application.

A method of controlling an insect population is also provided by the invention. The method generally involves contacting the population with an insecticidally-effective amount of a lipid as described herein. Such methods may be used to kill or reduce the numbers of target insects in a given area, or may be prophylactically applied to a locus, such as an environmental area, to prevent infestation by a susceptible insect.

In still a further aspect, the invention provides a method for producing a biological control composition, the method comprising:

providing a culture of one or more fungi of the phylum Ascomycota,

maintaining the culture under conditions suitable for production of at least one lipid of the invention; and
i) combining the at least one lipid of the invention with a carrier, or
ii) combining the at least one lipid of the invention with one or more entomopathogenic fungi described herein, or
iii) separating the at least one lipid of the invention from the fungi or fungal culture, or
iv) at least partially purifying or isolating the at least one lipid of the invention, or
v) any combination of two or more of (i) to (iv).

In one embodiment, the composition comprises a fungi, a fungal culture or a fungal culture supernatant as described above to which has been added a lipid of the invention.

The, invention further relates to the use of a lipid of the invention or a composition of the invention for the control one or more insects, such as one or more phytopathogenic insects. The use of a lipid of the invention in the manufacture of a composition for the control of one or more insects is similarly contemplated.

In a further aspect, the present invention provides a method of controlling one or more insects, the method comprising contacting the one or more insects with a lipid of the invention or a functional variant thereof.

The present invention further relates to a method for controlling one or more insects, such as one or more phytopathogenic insects, the method comprising applying to a locus, such as a plant or its surroundings, a lipid of the invention or a functional variant thereof, optionally together with at least one entomopathogenic fungus as described herein.

In another aspect, the present invention provides a method of reversing, wholly or in part, the resistance of an insect to one or more insecticides or one or more entomopathogenic agents, the method comprising contacting the insect with a lipid of the invention.

Optionally, the method comprises contacting the insect with a lipid or the invention together with one or more insecticides or one or more entomopathogenic agents, or any combination thereof.

In various embodiments, the one or more insecticides or one or more entomopathogenic agents administered is the same as that to which the insect is or is predicted to be or become resistant.

In a further aspect, the invention provides a method of controlling one or more insects which have been contacted with one or more lipids of the invention with an amount of an insecticide or entomopathogenic agent effective to control said one or more insects.

The one or more insecticides or one or more entomopathogenic agents may be administered prior to, concurrently with, or after administration of the lipid of the invention. Accordingly, administration of the one or more lipids of the invention and the one or more insecticides or entomopathogenic agents may be simultaneous, sequential, or separate.

In another aspect, the present invention provides a method of reversing, wholly or in part, the resistance of an insect to one or more insecticides or to one or more entomopathogenic agents, the method comprising contacting the one or more insects with an entomopathogenic fungi of the invention, optionally together with an insecticide or entomopathogenic agent.

In one embodiment, the method comprises contacting the one or more insects with an entomopathogenic fungi together with one or more lipids of the invention.

The following embodiments may relate to any of the aspects herein.

In one embodiment, the at least one fungus is of the class Sordariomycetes, including one or more fungi of the following subclasses:

Hypocreomycetidae, such as one or more fungi of the order Coronophorales, Hypocreales, Melanosporales, or Microascales; Sordariomycetidae, including one or more fungi of the order Boliniales, Calosphaeriales, Chaetosphaeriales, Coniochaetales, Diaporthales, Magnaporthales, Ophiostomatales, Sordariales; Xylariomycetida, including fungi of the order Xylariales and fungi of the order Koralionastetales, Lulworthiales, Meliolales, Phyllachorales, and Trichosphaeriales.

In one embodiment, the one or more fungi is of the order Hypocreales, such as for example one or more fungi of the family Bionectriaceae, Clavicipitaceae, Hypocreaceae, Nectriaceae, Niessliaceae, or Ophiocordycipitaceae.

In one embodiment, the one or more fungi is of the family Clavicipitaceae, including one or more fungi from the following genera:

Aciculosporium, Ascopolyporus, Atkinsonella, Atricordycep, Balansia, Berkelella, Cavimalum, Cepsiclava, Claviceps, Cordycepioideus, Cordyceps, Dussiella, Epichloé, Epicrea, Helminthascus, Heteroepichloë, Hyperdermium, Hypocrella, Konradia, Loculistroma, Metacordyceps, Moelleriella, Mycomalmus, Myriogenospora, Neobarya, Neoclaviceps, Neocordyceps, Parepichloe, Phytocordyceps, Podocrella, Regiocrella, Romanoa, Shimizuomyces, Sphaerocordyceps, Stereocrea, Torrubiella, Wakefieldiomyces, Akanthomyces, Aschersonia, Beauveria, Chaunopycnis, Corallocytostroma, Culicinomyces, Drechmeria, Ephelis, Gibellula, Haptocillium, Harposporium, Hirsutella, Hymenostilbe, Isaria, Lecanicillium, Mariannaea, Metarhizium, Microhilum, Neomunkia, Neotyphodium, Nomuraea, Paecilomyces, Pochonia, Polycephalomyces, Pseudogibellula, Simplicillium, Sorosporella, Tolypocladium or Ustilaginoidea.

For example, one or more fungi is of, the genus Beauveria, including for example one or more strains of Beauveria bassiana, Beauveria brongniartii, Beauveria felina, or Beauveria globulifera.

In another embodiment the one or more fungi is of the family Hypocreaceae, including one or more fungi from the following genera:

Aphysiostroma, Cladobotryum, Gliocladium, Hypocrea, Hypocreopsis, Hypomyces, Mycogone, Podostroma, Protocrea, Rogersonia, Sarawakus, Sepedonium, Sphaerostilbella, Sporophagomyces, Stephanoma or Trichoderma.

In one embodiment, the one or more fungi is of the genus Trichoderma including one or more of the following:

Trichoderma aggressivum, Trichoderma asperellum, Trichoderma atroviride Trichoderma aureoviride, Trichoderma austrokoningii, Trichoderma brevicompactum, Trichoderma candidum, Trichoderma caribbaeum var. aequatoriale, Trichoderma caribbaeum var. caribbaeum, Trichoderma catoptron, Trichoderma cremeum, Trichoderma ceramicum, Trichoderma cerinum, Trichoderma chlorosporum, Trichoderma chromospermum, Trichoderma cinnamomeum, Trichoderma citrinoviride, richoderma crassum, Trichoderma cremeum, Trichoderma dingleyeae, Trichoderma dorotheae, Trichoderma effusum, Trichoderma erinaceum, Trichoderma estonicum, Trichoderma fertile, Trichoderma gelatinosus, Trichoderma ghanense, Trichoderma hamatum, Trichoderma harzianum, Trichoderma helicum, Trichoderma intricatum, Trichoderma konilangbra, Trichoderma koningii, Trichoderma koningiopsis, Trichoderma longibrachiatum, Trichoderma longipile, Trichoderma minutisporum, Trichoderma oblongisporum, Trichoderma ovalisporum, Trichoderma petersenii, Trichoderma phyllostahydis, Trichoderma piluliferum, Trichoderma pleuroticola, Trichoderma pleurotum, Trichoderma polysporum, Trichoderma pseudokoningii, Trichoderma pubescens, Trichoderma reesei, Trichoderma rogersonii, Trichoderma rossicum, Trichoderma saturnisporum, Trichoderma sinensis, Trichoderma sinuosum, Trichoderma sp. MA 3642, Trichoderma sp. PPRI 3559, Trichoderma spirale, Trichoderma stramineum, Trichoderma strigosum, Trichoderma stromaticum, Trichoderma surrotundum, Trichoderma taiwanense, Trichoderma thailandicum, Trichoderma thelephoricolum, Trichoderma theobromicola, Trichoderma tomentosum, Trichoderma velutinum, Trichoderma virens, Trichoderma viride, Trichoderma viridescens; or one or more Hypocrea species, including Hypocrea phyllostachydis.

In various embodiments, the one or more fungi is one or more strains selected from Beauveria bassiana K4B3 NMIA No. V08/025855 or a strain having the identifying characteristics thereof; Lecanicillium muscarium strain K4V1 (NMIA No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

In various embodiments the composition or isolate obtained or obtainable from a fungus of the phylum Ascomycota comprises at least about 1, 2, 3, 4, 5, 6, 7, 8, 9, 10, 15, 20, 25, 30, 35, 40, 45, 50, 55, 60, 65, 70, 75, 80, 85, 90, 95 or 99% by weight of a lipid, and useful ranges may be selected between any of these values (for example, about 1 to about 99, about 5 to about 99, about 10 to about 99, about 15 to about 99, about 20 to about 99, about 25 to about 99, about 30 to about 99, about 35 to about 99, about 40 to about 99, about 45 to about 99, about 50 to about 99, about 55 to about 99, about 60 to about 99, about 65 to about 99, about 70 to about 99, about 75 to about 99, about 80 to about 99, about 85 to about 99, or about 90 to about 99% by weight).

It should be understood that any compositions or isolates useful herein include compositions and isolates obtained or obtainable from a culture comprising one or more fungi of the phylum Ascomycota, and may be obtained from a culture in which one or more fungi of the phylum Ascomycota is or was present but has since been removed.

In one embodiment a composition useful herein comprises at least about, 0.001, 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 mg/mL of a lipid, and useful ranges may be selected between any of these values (for example, about 0.01 to about 1.0, about 0.01 to about 10, about 0.01 to about 20, about 0.01 to about 30, about 0.01 to about 40, about 0.01 to about 50, about 0.01 to about 60, about 0.01 to about 70, about 0.01 to about 80, about 0.01 to about 90, about 0.01 to about 100, about 0.1 to about 1.0, about 0.1 to about 10, about 0.1 to about 20, about 0.1 to about 30, about 0.1 to about 40, about 0.1 to about 50, about 0.1 to about 60, about 0.1 to about 70, about 0.1 to about 80, about 0.1 to about 90, about 0.1 to about 100, about 0.7 to about 1.0, about 0.7 to about 10, about 0.7 to about 20, about 0.7 to about 30, about 0.7 to about 40, about 0.7 to about 50, about 0.7 to about 60, about 0.7 to about 70, about 0.7 to about 80, about 0.7 to about 90, or about 0.7 to about 100 mg/mL).

Exemplary fungal cells that produce one or more lipids of the invention include Beauvaria bassiana strain K4B3 on deposit at National Measurement Institute of Australia (NM1A) under Accession No. V08/025855 deposited 14 Oct. 2008, or a culture having the identifying characteristics thereof; Beauvaria bassiana strain AM2, Beauvaria bassiana strain F480, Trichoderma isolate 1328, and Metarhizium sp, or strains having the identifying characteristics of any one thereof.

In one embodiment, the invention relates to a method of preparing an insecticidal lipid, the method comprising

providing an organic solvent extraction of a culture of one or more fungi of the phylum Ascomycota,

at least partially separating one or more lipids of the invention from one or more other compounds, and

recovering the one or more lipids.

In one embodiment, the invention relates to a method of preparing an insecticidal lipid, the method comprising

providing an organic solvent extraction of a culture of one or more fungi of the phylum Ascomycota,

at least partially separating one or more lipids of the invention from one or more other compounds, and

recovering the one or more lipids.

In certain embodiments, the method of preparing a lipid having insecticidal activity from a fungus of the phylum Ascomycota is essentially as herein described.

In one exemplary embodiment, the organic solvent is or comprises chloroform. For example, the organic solvent comprises methanol and chloroform. In another embodiment, the organic solvent is dichloromethane.

In various embodiments, the organic solvent is an alkanol including a short chain alkyl alcohol, such as but not limited to methanol, ethanol, propanol, iso-propanol, or butanol, or is chloroform.

In various embodiments, the organic solvent is an agriculturally acceptable carrier, including a carrier as described herein.

In various embodiments, the separation is by chromatography, including anion exchange chromatography and thin layer chromatography. In one exemplary embodiment, the anion exchange chromatography is with DEAE-Sephadex.

The composition of the invention may be formulated as a powder, dust, pellet, granule, spray, emulsion, colloid, solution, or such like, and may be preparable by such conventional means as desiccation, lyophilization, homogenization, extraction, filtration, centrifugation, sedimentation, or concentration of a culture of cells comprising the lipid. In some embodiments of exemplary compositions that contain at least one such insecticidal lipid, the lipid is present in a concentration of from about 1% to about 99% by weight.

Preferably such compositions are obtainable from one or more cultures of the B. bassiana cells described herein. An exemplary insecticidal lipid formulation may be prepared by a process comprising the steps of culturing a suitable Beauvaria bassiana strain K4B3 cell under conditions effective to produce mycelia, providing at least partially purified mycelia, and obtaining one or more lipids from the mycelia.

In a further embodiment, the invention provides methods for preparing an insecticidal lipid composition. In exemplary embodiments, such lipids may be formulated for use as an insecticidal agent, and may be used to control insect populations in an environment, including agricultural environs and the like. In some embodiments, the formulations can be used to kill an insect or insect population, either by topical application, or by ingestion of the lipid composition by the insect. In other embodiments, the formulations can be used to antagonise an insect or insect population, again either by topical application or by ingestion of the peptide composition by the insect(s). In certain instances, it may be desirable to formulate the lipids of the present invention for application to the soil, on or near plants, trees, shrubs, and the like, near live plants, livestock, domiciles, farm equipment, buildings, and the like.

In various embodiments of a composition comprising one or more lipids of the invention and one or more fungi, the one or more fungi is in a reproductively viable form and amount.

In one embodiment the invention provides a composition comprising one or more lipids as described herein and spores obtainable from a least one fungi together with at least one carrier.

Preferably, said composition is a biological control composition, more preferably said biological control composition is an insecticidal composition.

Preferably, said biological control composition comprises at least one agriculturally acceptable carrier.

Preferably, said at least one carrier is an agriculturally acceptable carriers, more preferably is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant.

Preferably, said filler stimulant is a carbohydrate source, such as a disaccharide including, for example, sucrose, fructose, glucose, or dextrose, said anti-caking agent is selected from talc, silicon dioxide, calcium silicate, or kaelin clay, said wetting agent is skimmed milk powder, said emulsifier is a soy-based emulsifier such as lecithin or a vegetable-based emulsifier such as monodiglyceride, and said antioxidant is sodium glutamate or citric acid.

In various embodiments, the composition is a stable composition capable of supporting reproductive viability of the fungi or capable of retaining insecticidal efficacy for a period greater than about two weeks, preferably greater than about one month, about two months, about three months, about four months, about five months, more preferably greater than about six months.

In certain embodiments, the composition comprises a single strain of fungus. In one exemplary embodiment, the fungus is Beauveria bassiana strain K4B3 (NMIA No. V08/025855 deposited 14 Oct. 2008).

Alternatively, the composition comprises multiple strains of said fungi. In one embodiment, the composition is a biological control composition that comprises one or more lipids of the invention, together with, in a reproductively viable form and amount one or more strains selected from Beauveria bassiana K4B3 NM1A No. V08/025855 or a strain having the identifying characteristics thereof; Lecanicillium muscarium strain K4V1 (NMIA No. NM05/44593 deposited 16 Mar. 2005) or a strain having the identifying characteristics thereof; Lecanicillium muscarium strain K4V2 (NMIA Accession No. NM05/44594 deposited 16 Mar. 2005) or a strain having the identifying characteristics thereof; Lecanicillium muscarium strain K4V4 (NMIA Accession No. NM06/00007 deposited 3 Mar. 2006) or a strain having the identifying characteristics thereof; Beauveria bassiana strain K4B1 (NMIA Accession No. NM05/44595 deposited 16 Mar. 2005) or a strain having the identifying characteristics thereof; Beauveria bassiana strain K4B2 (NMIA Accession No. NM06/00010 deposited 3 Mar. 2006) or a strain having the identifying characteristics thereof; Lecanicillium longisporum strain KT4L1 (NMIA Accession No. NM06/00009 deposited 3 Mar. 2006) or a strain having the identifying characteristics thereof; and Paecilomyces fumosoroseus strain K4P1 (NMIA Accession No. NM06/00008 deposited 3 Mar. 2006) or a strain having the identifying characteristics thereof, and at least one agriculturally acceptable carrier.

In one embodiment the method for producing a biological control composition comprises:

providing a culture of Beauveria bassiana K4B3 V08/025855,

maintaining the culture under conditions suitable for production of at least one lipid of the invention; and
i) combining the at least one lipid of the invention with a carrier, or
ii) combining the at least one lipid of the invention with one or more entomopathogenic fungi described herein, or
iii) separating the at least one lipid of the invention from the Beauveria bassiana K4B3 V08/025855, or
iv) any combination of two or more of (i) to (iii).

In certain embodiments, the method may additionally comprise after the maintaining step one or cell-lysis steps.

In various embodiments the separation is by centrifugation or by filtration.

In various embodiments, the separation is effective to remove greater than about 50% of the fungi, for example Beauveria bassiana K4B3 V08/025855, greater than about 55%, greater than about 60%, greater than about 65%, greater than about 70%, greater than about 75%, greater than about 80%, greater than about 85%, greater than about 90%, greater than about 95%, greater than about 99%, or about 100% of the fungi, for example about 100% of the Beauveria bassiana K4B3 V08/025855.

Accordingly, in one particularly contemplated embodiment, the method comprises providing a culture of Beauveria bassiana K4B3 V08/025855, maintaining the culture under conditions suitable for production of at least one lipid of the invention, and separating the at least one lipid of the invention from the Beauveria bassiana K4B3 V08/025855.

Preferably, the carrier is an agriculturally acceptable carrier, preferably the at least one carrier is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant.

In various embodiments the phytopathogenic insect is of the order Hemiptera.

Preferably, said one or more phytopathogenic insects is selected from the group consisting of mosquito, moths including diamond back moth, Thrips (Thysanoptera), Aphids, Psyllids, Scale or Whitefly (Hemiptera).

In one example, the lipid of the invention or functional variant thereof may be present in a composition as described herein.

In one embodiment, the composition comprises two or more lipids of the invention.

In various embodiments, the composition comprises at least one lipid of the invention, together with:

i) at least one beauvericin;

ii) at least one bassianolide;

iii) at least one entomopathogenic fungi

iv) any two or more of (i) to (iv) above.

In one embodiment, the present invention provides a method for controlling one or more insects, such as one or more phytopathogenic insects, the method comprising applying to a locus, such as a plant or its surroundings, a lipid or composition of the present invention.

In one embodiment, the method for controlling one or more insects comprises applying to the locus a lipid of formula I

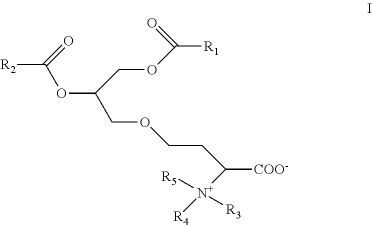

wherein R₁ and R₂, which can be the same or different, are each the aliphatic portion of a fatty acid, and wherein R3, R4 and R5 are each independently selected from the group comprising or consisting of H, hydroxyl, carboxyl, amide, unsubstituted or substituted alkyl, unsubstituted or substituted alkenyl, unsubstituted or substituted alkynyl, and unsubstituted or substituted aryl moieties.

In various embodiments, the lipid or composition of the invention is applied prophylactically, for example before the locus, such as a plant, is infected by or exposed to the insect or insect population. In other embodiments, the composition is applied when infection is established or the pathogen is present, for example when a locus such as a plant is infected by or exposed to an insect, or when an insect is present on or in the locus.

In one embodiment, lipids or compositions of the invention are applied directly to the locus, for example are applied directly to a plant or its surroundings. For example, a composition of the invention is admixed with a solvent or emulsified (for example with water) and applied as described herein. In other embodiments, the lipids or compositions of the invention are applied indirectly to the locus, such as for example by application to a substrate that is subsequently applied to the locus.

Preferably, the composition is admixed with water to a final concentration of lipid of about 0.5 gm/L to about 10 gm/L prior to application, and more preferably to a final, concentration of about 1 gm/L.

Preferably, a desiccation protection agent, such as Deep Fried™, Fortune™, or Fortune Plus™, is admixed to a final concentration of about 1 ml/L, prior to application.

For compositions comprising one or more entomopathogenic fungi, an exemplary concentration range is from about 1×10² to about 1×10¹² spores per ml, from about 1×10² to about 1×10¹¹ spores per ml, from about 1×10² to about 1×10¹⁰ spores per ml, from about 1×10² to about 1×10⁹ spores per ml, from about 1×10³ to about 1×10⁹ spores per ml, from about 1×10⁴ to about 1×10⁹ spores per ml, preferably from about 1×10⁵ to about 5×10⁸, and more preferably about 1×10⁶ to about 2×10⁸ spores per ml. In certain embodiments, the composition comprises at least 10⁷ spores per millilitre at application, at least about 5×10⁷ spores per millilitre at application, or at least 10⁸ spores per ml at application.

In various embodiments when one or more fungi are present in the composition, the compositions of the invention may be applied at a rate of from about 1×10⁸ to about 1×10¹⁵ infectious units (IU) per hectare, from about 1×10⁹ to about 1×10¹⁵ IU per hectare, from about 1×10¹⁰ to about 1×10¹⁵ IU per hectare, from about 1×10¹¹ to about 1×10¹⁵ IU per hectare, preferably from about 1×10¹¹ to about 1×10¹⁴ IU per hectare, more preferably from about 5×10¹⁰ to about 1×10¹⁴ IU per hectare, more preferably about 1×10¹¹ to about 5×10¹¹ IU per hectare.

In one embodiment, the infectious unit is a spore, such as an endospore, and the composition is applied at a rate of from about 1×10⁸ to about 1×10¹⁵ spores per hectare, from about 1×10⁹ to about 1×10¹⁵ spores per hectare, from about 1×10¹⁰ to about 1×10¹⁵ spores per hectare, from about 1×10¹¹ to about 1×10¹⁵ spores per hectare, preferably from about 1×10¹⁰ to about 1×10¹⁴ spores per hectare, more preferably from about 5×10¹⁰ to about 1×10¹⁴ spores per hectare, more preferably about 1×10¹¹ to about 5×10¹¹ spores per hectare.

Conveniently, such a rate of application can be achieved by formulating said composition at about 10⁸ spores per millilitre or more, and applying said composition at a rate of about 1 L per hectare. As discussed herein, such an application rate can be conveniently achieved by dissolution of the composition in a larger volume of agriculturally acceptable solvent, for example, water.

Preferably, the composition is admixed with water prior to application. In one embodiment, the composition is admixed with water and applied in at least about 100 L water/Ha, in at least about 150 L/Ha, in at least about 200 L/Ha, in at least about 250 L/Ha, in at least about 300 L/Ha, in at least about 350 L/Ha, in at least about 400 L/Ha, in at least about 450 L/Ha, or in at least about 500 L/Ha. In a preferred embodiment, the composition is admixed with water to a final concentration of about 1×10¹¹ to about 5×10¹¹ spores per 500 L water prior to application and applied at a rate of 500 L/hectare.

Preferably, said application is by spraying.

Preferably, a composition comprising Beauveria bassiana strain K4B3 (NMIA Accession No. V08/025855) or a culture having the identifying characteristics thereof is applied at a rate of from about 1×10¹⁰ to about 1×10¹⁵ spores per hectare, preferably from about 1×10¹² to about 1×10¹⁴ spores per hectare, more preferably from about 5×10¹² to about 1×10¹⁴ spores per hectare, more preferably about 1-3×10¹³ spores per hectare.

Conveniently, such a rate of application can be achieved by formulating said composition at about 10⁷ spores per milligram or more, and applying said composition at a rate of about 1 kg per hectare. As discussed herein, such an application rate can be conveniently achieved by dissolution of the composition in a larger volume of agriculturally acceptable solvent, for example, water.

The invention is applicable to any plant or its surroundings. Exemplary plants are in certain embodiments monocotyledonous or dicotyledonous plants such as alfalfa, barley, canola, corn, cotton, flax, kapok, peanut, potato, oat, rice, rye, sorghum, soybean, sugarbeet, sugarcane, sunflower, tobacco, tomato, wheat, turf grass, pasture grass, berry, fruit, legume, vegetable, ornamental plants, shrubs, cactuses, succulents, and trees.

In further illustrative embodiments, the plant may be any plant, including plants selected from the order Solanales, including plants from the following families: Convolvulaceae, Hydroleaceae, Montiniaceae, Solanaceae, and Sphenocleaceae, and plants from the order Asparagales, including plants from the following families: Amaryllidaceae, Asparagaceae, Asteliaceae, Blandfordiaceae, Boryaceae, Doryanthaceae, Hypoxidaceae, Iridaceae, Ixioliriaceae, Lanariaceae, Orchidaceae, Tecophilaeaceae, Xanthorrhoeaceae, and Xeronemataceae.

To those skilled in the art to which the invention relates, many changes in construction and differing embodiments and applications of the invention will suggest themselves without departing from the scope of the invention as defined in the appended claims. The disclosures and the descriptions herein are purely illustrative and are not intended to be in any sense limiting.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is intended that reference to a range of numbers disclosed herein (for example, 1 to 10) also incorporates reference to all rational numbers-within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### DESCRIPTION OF FIGURES

FIG. 1 shows a chromatogram of exemplary betaine lipids as described in Example 4 herein.

FIG. 2 shows an MS/MS spectrum of exemplary betaine lipids as described in Example 4 herein.

FIG. 3 shows a daughter spectrum of exemplary lipid having m/z of 762.5 as described in Example 4 herein.

FIG. 4 shows the relative amounts of the exemplary betaine lipids produced over a 10-day time course, as described in Example 6 herein.

### DETAILED DESCRIPTION OF THE INVENTION

The present invention is in part directed to one or more lipids, including one or more lipids isolated from various strains of Ascomycota fungi, such as Beauveria spp. and Trichoderma spp., wherein the one or more lipids have efficacy against insects, such as phytopathogenic insects, and the use of such lipids in controlling insects and insect populations.

### DEFINITIONS

As used herein, the term “betaine lipid” means an ether-linked glycerolipid containing a betaine moiety linked by an ether bond at the sn-3 position of the glycerol moiety, with fatty acids esterified in the sn-1 and sn-2 positions. Betaine lipids have been reported to occur in algae, bryophytes, fungi, some primitive protozoa, photosynthetic bacteria and in some spore-producing plants, such as ferns and species belonging to the Equisetophyta.

The term “biological control agent” (BCA) as used herein refers to a biological agent which acts as an antagonist of one or more organisms, typically one or more pests or pathogens such as one or more phytopathogens, such as a phytopathogenic insect, or is able to control one or more one or more pathogens such as one or more phytopathogens. Antagonism may take a number of forms. In one form, the biological control agent may simply act as a repellent. In another form, the biological control agent may render the environment unfavourable for the pathogen. In a further, preferred form, the biological control agent may parasitise, incapacitate, render infertile, impede the growth of, impede the spread or distribution of, and/or kill the pest or pathogen. Accordingly, the antagonistic mechanisms include but are not limited to antibiosis, parasitism, immobilisation, infertility, and toxicity. Therefore, agents which act as antagonists of one or more pathogenic insects can be said to have insecticidal efficacy. Furthermore, an agent that is an antagonist of an insect, including a phytopathogenic insect, can be said to be an insecticidal agent.

As used herein, a “biological control composition” is a composition comprising or including at least one biological control agent that is an antagonist of one or more pests or pathogens, such as one or more plant pests or phytopathogens. Such control agents include, but are not limited to, agents that act as repellents, agents that render the environment unfavourable for the pest or pathogen, and agents that incapacitate, render infertile, and/or kill the pest or pathogen.

Accordingly, as used herein an “anti-phytopathogenic composition” is a composition which comprises or includes at least one agent that is an antagonist of one or more phytopathogens. Such a composition is herein considered to have anti-phytopathogenic efficacy.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “control” or “controlling” as used herein generally comprehends preventing, reducing, or eradicating infection by one or more pathogens such as infection by one or more phytopathogens, or inhibiting the rate and extent of such infection, such as reducing a phytopathogen population in or on a plant or its surroundings, wherein such prevention or reduction in the infection(s) or population(s) is statistically significant with respect to untreated infection(s) or population(s). Curative treatment is also contemplated. Preferably, such control is achieved by increased mortality amongst the pathogen population.

The phrases “entomopathogenic activity” and “entomopathogenic efficacy” are used interchangeably herein and refer to the ability of certain agents, such as certain microorganisms or agents derived from certain microorganisms, to antagonise one or more pathogenic insects, such as one or more phytopathogenic insects.

In various embodiments, said entomopathogenic efficacy is the ability to parasitise and incapacitate, render infertile, impede the growth of, or kill one or more insects, such as phytopathogenic insects, preferably within 14 days of contact with the insect, more preferably within 7 days, more preferably still the ability to kill one or more phytopathogenic insects within 7 days. Alternatively, said entomopathogenic efficacy is the ability to support or promote the growth of one more entomopathogenic microorganisms, such as one or more entomopathogenic fungi.

Accordingly, as used herein an “entomopathogenic composition” is a composition which comprises or includes at least one agent that is an antagonist of one or more pathogenic insects. Such a composition is herein considered to have entomopathogenic efficacy.

Accordingly, as used herein an “insecticidal composition” is a composition which comprises or includes at least one agent that is an antagonist of one or more pathogenic insects. Such a composition is herein considered to have insecticidal efficacy.

In certain embodiments, the entomopathogenic activity is insecticidal activity. For example, certain embodiments of the lipid of the invention are insecticidal.

The term “functional variant” as used herein in reference to one or more lipids, for example in respect of one or more lipids as exemplified herein in the Examples, refers to a lipid different from the specifically identified entity, for example wherein one or more groups, such as one or more fatty acid groups is deleted, substituted, or added, but which possesses at least in part one or more of the biological activities of the specifically-identified entity, such as an ability to elicit one or more biological effects elicited by the specifically-identified lipid. Functional variants may be from the same or from other species and may encompass homologues, paralogues and orthologues.

In the present case, the functional variant will preferably retain at least a portion of the insecticidal activity of the specifically-identified lipid.

Methods and assays to determine one of more biological effects elicited by the lipids of the invention, such as insecticidal efficacy, are well known in the art, and such methods and assays can be used to identify or verify one or more functional variants of one or more of the lipids of the invention. For example, an assay of the ability of a lipid of the invention to kill or otherwise antagonise the growth of a target insect, such as those described herein in the Examples, is amenable to identifying one or more functional variants of the lipid.

The term “lipid”, as used herein, encompasses highly reduced carbon-rich substances that are insoluble in water and comprise one or more fatty acids, carboxylic acids with hydrocarbon chains typically ranging from 4 to 36 carbon atoms in length. Lipids include triacylglycerols, phospholipids including glycerophospholipids and sphingolipids, glycolipids, and sterols.

The term “lipid fraction”, as used herein, encompasses a composition comprising one or more lipids, free fatty acids, or both, wherein the fraction comprises or consists of a subset of total lipids present in the unfractionated source material. Typically, lipid fractions will have a determinable and identifiable composition, for example a characteristic chromatography profile or mass spectragraphic profile. Examples of lipid fractions are presented herein.

The term “plant” as used herein encompasses not only whole plants, but extends to plant parts, cuttings as well as plant products including roots, leaves, flowers, seeds, stems, callus tissue, nuts and fruit, bulbs, tubers, corms, grains, cuttings, root stock, or scions, and includes any plant material whether pre-planting, during growth, and at or post harvest. Plants that may benefit from the application of the present invention cover a broad range of agricultural and horticultural crops. The compositions of the present invention are also especially suitable for application in organic production systems.

When used in respect of an entomopathogenic agent, such as an entomopathogenic lipid or an entomopathogenic fungal strain, the phrase “retaining entomopathogenic efficacy” and grammatical equivalents and derivatives thereof is intended to mean that the agent still has useful entomopathogenic activity. Preferably, the retained activity is at least about 35, 40, 45, 50, 55, 60, 65, 70, 75, 80, 85, 90, 95, 99 or 100% of the original activity, and useful ranges may be selected between any of these values (for example, from about 35 to about 100%, from about 50 to about 100%, from about 60 to about 100%, from about 70 to about 100%, from about 80 to about 100%, and from about 90 to about 100%). For example, preferred lipid functional variants of the present invention should retain entomopathogenic activity, that is, retain at least about 35, 40, 45, 50, 55, 60, 65, 70, 75, 80, 85, 90, 95, 99 or 100% of the entomopathogenic activity of the specified parent lipid. Similarly, preferred compositions of the invention are capable of supporting the maintenance of useful entomopathogenic activity of the entomopathogenic agent(s) they comprise, and can be said to retain entomopathogenic activity, ideally until applied using the methods contemplated herein.

Similarly, when used in respect of an insecticidal agent, such as an insecticidal lipid or an entomopathogenic fungal strain, the phrase “retaining insecticidal efficacy” and grammatical equivalents and derivatives thereof is intended to mean that the agent still has useful insecticidal activity. Preferably, the retained activity is at least about 35, 40, 45, 50, 55, 60, 65, 70, 75, 80, 85, 90, 95, 99 or 100% of the original activity, and useful ranges may be selected between any of these values (for example, from about 35 to about 100%, from about 50 to about 100%, from about 60 to about 100%, from about 70 to about 100%, from about 80 to about 100%, and from about 90 to about 100%). For example, preferred lipid functional variants of the present invention should retain insecticidal activity, that is, retain at least about 35, 40, 45, 50, 55, 60, 65, 70, 75, 80, 85, 90, 95, 99 or 100% of the insecticidal activity of the specified parent lipid. Similarly, preferred compositions of the invention are capable of supporting the maintenance of useful insecticidal activity of the insecticidal agent(s) they comprise, and can be said to retain insecticidal activity, ideally until applied using the methods contemplated herein.

As used herein, the term “stable” when used in relation to a composition of the invention means a composition capable of supporting insecticidal efficacy for several weeks, preferably about one, about two, about three, about four, preferably about five, more preferably about six months, or longer. For example, when used in relation to a composition additionally comprising one or more entomopathogenic fungi, the term “stable” refers to a composition capable of supporting reproductive viability of the entomopathogenic fungi for several weeks, preferably about one, about two, about three, about four, preferably about five, more preferably about six months or longer.

A “strain having the identifying characteristics of [a specified strain]”, or a “culture having the identifying characteristics of [a specified culture]” including a homologue or mutant of the specified strain, is closely related to (i.e., shares a common ancestor with) or is derived from the specified strain, but will usually differ from the specified strain in one or more genotypic or phenotypic characteristics. Mutants are generally identifiable through assessment of genetic differences. Homologues are identifiable through assessment of the degree of genetic, biochemical and morphological difference and use of taxonomic methods, including for example analyses such as cladistics. However, a strain having the identifying characteristics of [a specified strain], including a homologue or mutant of the specified strain will retain insecticidal efficacy, will be distinguishable from other bacterial strains, and will be identifiable as a homologue or mutant of the parent strain using the techniques described herein.

The term “surroundings” when used in reference to a plant subject to the fungi, methods and compositions of the present invention includes soil, water, leaf litter, and/or growth media adjacent to or around the plant or the roots, tubers or the like thereof, adjacent plants, cuttings of said plant, supports, water to be administered to the plant, and coatings including seed coatings. It further includes storage, packaging or processing materials such as protective coatings, boxes and wrappers, and planting, maintenance or harvesting equipment.

Control of Phytopathogens

The present invention recognises that the horticultural sectors of many countries, including for example that of the United States of America, of New Zealand, and many states of Europe, are faced with the problem of increasing insecticide resistance amongst phytopathogenic insect pests. This is compounded under some regulatory regimes by a reduction in the availability of new chemical insecticides due to regulatory barriers.

The use of insecticidal lipids derived from fungi as biological control agents presents a solution to this problem. Effective biological control agents can be selected according to their ability to incapacitate or kill a target phytopathogenic insect or insect population. Under conducive conditions, phytopathogenic insects such as aphids, thrips and whitefly may infect plants and their surroundings including soil, leaf litter, adjacent plants, supports, and the like. Insecticidal lipids derived from fungi and agents derived therefrom may be applied so as to incapacitate and/or kill the phytopathogenic insect, thereby preventing or limiting the disease-causing capability of the pathogen. The effectiveness of these insecticidal lipids derived from fungi in the field is partly due to their ability to retain insecticidal efficacy in varying climatic conditions, such as interrupted wet periods and desiccation. The effectiveness of agents derived from entomopathogenic fungi such as the lipids described herein does not typically require a maintenance of viability, but rather a maintenance of insecticidal efficacy. Again, as will be appreciated, the stability of the composition, such as for example a resistance to colonisation by contaminating microorganisms, an ability to suppress or not support the growth of microorganisms, or a bacteriostatic or fungistatic character, may all contribute to the utility of the composition and/or a maintenance of molluscicidal efficacy.

A lipid of the invention, effective against insects, such as phytopathogenic insects, and therefore suitable for use in accordance with the invention, is identified as one which is effective at reducing the population of the target insect species by a statistically significant amount with respect to the control treatment against which the lipids of the invention or functional variants thereof is compared. Such lipids can be considered as having insecticidal efficacy. As described herein, the reduction in the population of the target insect may be by various antagonistic mechanisms. For example, the lipid may incapacitate, render infertile, inhibit the growth or development of, and/or preferably kill the phytopathogenic insect, or may support or promote the growth and insecticidal efficacy of one or more entomopathogens also present, such as an entomopathogenic fungi present in a composition together with the lipid of the invention (whether separately, simultaneously, or sequentially). As such, the lipids of the invention may enable or support the ability of the entomopathogen such as an entomopathogenic fungi to parasitise, incapacitate, render infertile, and/or preferably kill the phytopathogenic insect. The lipids of the invention may also reduce the population of the target insect by rendering the environment, for example the plant to which the one or more fungi is applied or its surroundings, unfavourable for the phytopathogenic insect. In this embodiment, the lipid may be considered to be acting as a repellent, and reducing the effective population of the target insect in the vicinity of the plant or its surroundings.

In one embodiment the lipid is a functional variant as defined herein.

Preferably, suitable lipids of the invention or functional variants thereof exhibit about 5% insecticidal efficacy, at least about 10%, at least about 15%, at least about 20%, at least about 25%, at least about 30%, at least about 35%, at least about 40%, at least about 45%, more preferably at least about 50% insecticidal efficacy expressed as a percentage reduction of the population of the relevant insect species compared to the control treatment. By way of illustration, the methodology described herein was employed to identify Beauveria lipid fractions isolate effective against a variety of target insects, whereas procedures analogous to those described herein can be employed in relation to other fungi and insect species.

Although insecticidal efficacy is a principal requisite for a lipid to be considered suitable for use as a biological control agent, the lipid may have additional characteristics to be suitable for use as a biological control agent.

For example, the lipid should be able to be stored in effective form for a reasonable period, ultimately so as to allow it to be applied to the target plant or its surroundings in a form and concentration that is effective as a biological control agent.

Those skilled in the art will recognise that the lipids and compositions of the invention may comprise or the methods of the invention may use one or more functional variants of one or more of the lipids of the invention including those exemplified herein. Combinations of lipids and functional variants thereof are also useful herein.

Methods for Isolating Lipids

Exemplary methods to produce and isolate one or more of the lipids or of the invention are described herein. These include isolation of one or more lipids from a culture of one or more fungi of the phylum Ascomycota, such as for example a Beauveria species including for example B. bassiana K4B3 or a Trichoderma species.

Alternatively, the lipids of the invention, including functional variants thereof, may be prepared using lipid synthesis methods well known in the art.

Compositions Comprising Entomopathogenic Fungi

The importation of entomopathogenic fungi is frequently problematic, costly, and impractical if not impossible under certain regulatory regimes. For example, entomopathogenic fungi available outside a given country may not be available to horticulturalists within that country because of regulatory and legislative preclusions. The present invention therefore recognises there are distinct advantages to identifying and preparing agents that have insecticidal efficacy from such fungi, or are able to support or promote the growth of entomopathogenic fungi (or other entomopathogens) and so may be able to help such entomopathogens flourish under a wide variety of environmental conditions, or both.

Isolates of said fungi may conveniently be obtained from the environment, including, for example, from plants, their surroundings, and from pathogens of said plants. In certain embodiments, isolates of said fungi may be obtained from the target insect, or from the plant species (or surroundings) to which the biological control agent comprising said fungi or a composition comprising said fungi will subsequently be applied.

Methods to determine growth of said fungi under different conditions, including different temperatures and on different media or other substrates, are well known in the art. Likewise, methods to determine a positive impact on growth of fungi of a lipid of the invention, such as an increase in virulence or insecticidal efficacy of said fungi in the presence of the lipid(s), compared to that observed in the absence of said lipid(s) are also well known in the art. Examples of methods to determine the ability of a lipid of the invention to influence growth of fungi at various temperatures or on various plants, environments, or target organisms, are described herein.

Although entomopathogenic efficacy is a principal requisite for an isolate to be considered suitable for use as a biological control agent, the fungal isolate should have additional characteristics to be suitable for use as a biological control agent.

For example, the fungi should be able to be stored in a viable form for a reasonable period, ultimately so as to allow it to be applied to the target plant or its surroundings in a form and concentration that is effective as a biological control agent.

The fungi should also be able to achieve infection threshold when applied to a plant or its surroundings for it to be suitable for use as a biological control agent. As used herein, infection threshold refers to the concentration of fungi required for the fungi to become established on the target plant or its surroundings so as to then have entomopathogenic efficacy. As will be appreciated, in order to achieve infection threshold, some isolates of fungi may require application at such a high rate as to be impractical or unviable. Furthermore, some fungal isolates may not be, able to achieve infection threshold irrespective of the concentration or rate at which they are applied. Suitable entomopathogenic fungi are able to achieve infection threshold when applied at a rate of not less that 10¹⁰ spores per hectare, or applied at a concentration not less than 10⁷ spores per milligram of composition when said composition is applied at a rate of about 1 kg/1000 L/hectare.

Methods to determine infection threshold are well known in the art, and examples of such methods are presented herein. In certain embodiments, infection threshold can be determined directly, for example by analysing one or more samples obtained from a target plant, its surroundings, and/or a pathogen of said plant, and determining the presence or amount of fungus on or in said sample. In other embodiments, infection threshold can be determined indirectly, for example by observing a reduction in the population of one or more insects at a locus, such as a reduction in a population of phytopathogenic insects on or around a plant. Combinations of such methods are also envisaged.

Beauveria bassiana is a soil born fungi that attacks both immature and adult insects including, for example, grasshoppers, aphids, thrips, moths, and several other species. Typically, B. bassiana can be isolated from insect cadavers, such as aphids, borers, and thrips, and may also be isolated from soil. The exemplary insecticidal Beauveria bassiana strain K4B3 may be used in certain embodiments of the invention, and was deposited in the National Measurement Institute of Australia (NMIA, formerly the Australian Government Analytical Laboratories (AGAL)), 1 Suakin Street, Pymble, New South Wales, Australia on 14 Oct. 2008 according to the Budapest Treaty for the purposes of patent procedure. The isolate has been accorded the deposit number V08/025855.

Accordingly, in one aspect the present invention provides methods and compositions comprising reliant on one or more lipids of the invention, or one or more polynucleotides encoding them, together with a reproductively viable form and amount of B. bassiana strain K4B3, NMIA No. V08/025855, or Beauveria having the identifying characteristics of strain K4B3, NMIA No. V08/025855.

B. bassiana strain K4B3 is a particularly effective biological control agent, being capable of surviving interrupted wet periods, desiccation, and colonising, incapacitating and killing phytopathogenic insects such as, but not limited to, aphids, caterpillars, whitefly, moths, Varroa mite, cicada, and thrips in the field.

In other embodiments of the present invention, B. bassiana K4B3 may be used to prepare a composition comprising one or more lipids as identified herein.

In one embodiment, the method comprises maintaining a culture of Beauveria bassiana K4B3 V08/025855 under conditions suitable for production of at least one lipid; and separating the at least one lipid from the Beauveria bassiana K4B3 V08/025855.

In one embodiment, the composition comprises two or more lipids as described herein. Notably, none of these lipids has significant identity or homology to known insecticidal agents for Beauveria, such as beauvericin, and bassianolide. In one embodiment, the composition is a synergistic composition comprising two or more lipids as described herein.

In another embodiment, the composition additionally comprises less than about 1 mgL⁻¹ of a beauvericin, less than about 0.5 mgL⁻¹ of a beauvericin, less than about 0.1 mgL⁻¹ of a beauvericin, less than about 0.05 mgL⁻¹ of a beauvericin, less than about 0.01 mgL⁻¹ of a beauvericin, less than about 0.005 mgL⁻¹ of a beauvericin, less than about 0.001 mgL⁻¹ of a beauvericin, less than about 0.0005 mgL⁻¹ of a beauvericin, or less than about 0.0001 mgL⁻¹ of a beauvericin.

For example, the composition additionally comprises less than about 1 mgL⁻¹ beauvericin-F, less than about 0.5 mgL⁻¹ beauvericin-F, less than about 0.1 mgL⁻¹ beauvericin-F, less than about 0.05 mgL⁻¹ beauvericin-F, less than about 0.01 mgL⁻¹ beauvericin-F, less than about 0.005 mgL⁻¹ beauvericin-F, less than about 0.001 mgL⁻¹ beauvericin-F, less than about 0.0005 mgL⁻¹ beauvericin-F, or less than about 0.0001 mgL⁻¹ beauvericin-F.

In another embodiment, the composition additionally comprises less than about 1 mgL⁻¹ of a bassianolide, less than about 0.5 mgL⁻¹ of a bassianolide, less than about 0.1 mgL⁻¹ of a bassianolide, less than about 0.05 mgL⁻¹ of a bassianolide, less than about 0.01 mgL⁻¹ of a bassianolide, less than about 0.005 mgL⁻¹ of a bassianolide, less than about 0.001 mgL⁻¹ of a bassianolide, less than about 0.0005 mgL⁻¹ of a bassianolide, or less than about 0.0001 mgL⁻¹ of a bassianolide.

In various embodiments, the composition is a synergistic composition comprising one or more lipids as described herein, together with one or more beauvericin, such as beauvericin-A, beauvericin-D, beauvericin-E, or beauvericin-F, or with one or more bassianolide, or any combination thereof.

For example, the composition additionally comprises more than about 0.1 mgL⁻¹ of a beauvericin, more than about 0.5 mgL⁻¹ of a beauvericin, more than about 1 mgL⁻¹ of a beauvericin, more than about 5 mgL⁻¹ of a beauvericin, more than about 10 mgL⁻¹ of a beauvericin, more than about 50 mgL⁻¹ of a beauvericin, or more than about 100 mgL⁻¹ of a beauvericin.

In another example, the composition additionally comprises more than about 0.1 mgL⁻¹ of a bassianolide, more than about 0.5 mgL⁻¹ of a bassianolide, more than about 1 mgL⁻¹ of a bassianolide, more than about 5 mgL⁻¹ of a bassianolide, more than about 10 mgL⁻¹ of a bassianolide, more than about 50 mgL⁻¹ of a bassianolide, or more than about 100 mgL⁻¹ of a bassianolide.

In another embodiment, the composition is a synergistic composition comprising one or more lipids as described herein and one or more entomopathogenic fungi as described herein, together with one or more beauvericin, such as beauvericin-A, beauvericin-D, beauvericin-E, or beauvericin-F, or with one or more bassianolide, or any combination thereof.

In a further embodiment, the composition is a synergistic composition comprising one or more lipids as described herein and one or more entomopathogenic fungi as described herein.

Beauveria bassiana strain K4B3 of the invention may be used singly, or in combination with other entomopathogenic fungi described herein. Examples of other entomopathogenic fungi are described in more detail below.

Beauveria bassiana strain K4B1 was isolated from a borer larva within a pine forest in Bombay, New Zealand. This B. bassiana isolate has been deposited in the National Measurement Institute of Australia, 1 Suakin Street, Pymble, New South Wales, Australia on 16 Mar. 2005 according to the Budapest Treaty for the purposes of patent procedure. The isolate has been accorded the deposit number NM05/44595.

Beauveria bassiana isolate K4B1 shows a preference for thrips adults, and is also pathogenic to thrip juveniles and pupae, aphids and whitefly. The conidia of K4B1 form cream aggregations.

Beauveria bassiana isolate K4B2 was isolated from a Lepidoptera caterpillar on a sunflower in the Aka Aka flats, New Zealand. This B. bassiana isolate has been deposited in the National Measurement Institute of Australia on 3 Mar. 2006 according to the Budapest Treaty for the purposes of patent procedure. The isolate has been accorded the deposit number NM06/00010.

Beauveria bassiana isolate K4B2 exhibits a preference for caterpillars, including soybean looper caterpillar and white butterfly and army worm caterpillar. This isolate is also pathogenic to thrip juveniles, adults, and pupae, aphids and whitefly. The conidia of K4B2 form yellow dusty aggregations.

NMIA No. V08/025855, NMIA No. NM05/44595, NMIA No. NM06/00010 and other suitable isolates of B. bassiana may be used in combination with one or more lipids of the invention or functional variants or fragments thereof, and are particularly effective biological control agents; being capable of surviving interrupted wet periods, desiccation, and colonising, incapacitating and killing phytopathogenic insects such as, but not limited to, aphids, caterpillars, whitefly, moths, Varroa mite and thrips in the field. The degree of killing of whitefly, thrips and aphids by these isolates of B. bassiana is generally as good as the commonly used insecticides as described above. Resistance to these insecticides has developed; in these and other instances, compositions comprising or methods utilising B. bassiana isolates provide an effective alternative for insect control. This potent activity in the control of plant disease coupled with the absence of any observations of plant pathogenicity induced by B. bassiana demonstrate that isolates of these species have desirable attributes for use as a biological control agent.

Trichoderma species have not previously been ascribed any insecticidal activities or effects. Trichoderma cultures are typically fast growing at 25-30° C., but will not grow at 35° C. Colonies are transparent at first on media such as cornmeal dextrose agar (CMD) or white on richer media such as potato dextrose agar (PDA). Mycelium are not typically obvious on CMD, conidia typically form within one week in compact or loose tufts in shades of green or yellow or less frequently white. A yellow pigment may be secreted into the agar, especially on PDA. Some species produce a characteristic sweet or ‘coconut’ odor.

Conidiophores are highly branched and thus difficult to define or measure, loosely or compactly tufted, often formed in distinct concentric rings or borne along the scant aerial hyphae. Main branches of the conidiophores produce lateral side branches that may be paired or not, the longest branches distant from the tip and often phialides arising directly from the main axis near the tip. The branches may rebranch, with the secondary branches often paired and longest secondary branches being closest to the main axis. All primary and secondary branches arise at or near 90° with respect to the main axis. The typical Trichoderma conidiophore, with paired branches assumes a pyramidal aspect. Typically the conidiophore terminates in one or a few phialides. In some species (e.g. T. polysporum) the main branches are terminated by long, simple or branched, hooked, straight or sinuous, septate, thin-walled, sterile or terminally fertile elongations. The main axis may be the same width as the base of the phialide or it may be much wider.

Phialides are typically enlarged in the middle but may be cylindrical or nearly subglobose. Phialides may be held in whorls, at an angle of 90° with respect to other members of the whorl, or they may be variously penicillate (gliocladium-like). Phialides may be densely clustered on wide main axis (e.g. T. polysporum, T. hamatum) or they may be solitary (e.g. T. longibrachiatum).

Conidia typically appear dry but in some species they may be held in drops of clear green or yellow liquid (e.g. T. virens, T. flavofuscum). Conidia of most species are ellipsoidal, 3-5×2-4 μm (L/W=>1.3); globose conidia (L/W<1.3) are rare. Conidia are typically smooth but tuberculate to finely warted conidia are known in a few species.

Synanamorphs are formed by some species that also have typical Trichoderma pustules. Synanamorphs are recognized by their solitary conidiophores that are verticillately branched and that bear conidia in a drop of clear green liquid at the tip of each phialide.

Chlamydospores may be produced by all species, but not all species produce chlamydospores on CMD at 20° C. within 10 days. Chlamydospores are typically unicellular subglobose and terminate short hyphae; they may also be formed within hyphal cells. Chlamydospores of some species are multicellular (e.g. T. stromaticum).

Lecanicillium muscarium is an entomopathogenic fungi with a broad host range including homopteran insects and other arthropod groups. L. muscarium is considered a species complex, which includes isolates of varied morphological and biochemical characteristics. Typically, L. muscarium can be isolated from insect cadavers, such as aphids, thrips, whitefly, and mealy bugs, and may also be isolated from soil.

Lecanicillium muscarium strain K4V1 was isolated from whitefly in a greenhouse tomato crop in Pukekohe, New Zealand. This L. muscarium isolate has been deposited in the National Measurement Institute of Australia on 16 Mar. 2005 according to the Budapest Treaty for the purposes of patent procedure. The isolate has been accorded the deposit number NM05/44593.

K4V1 has the additional identifying characteristics—60% Conidia 1.0×1.0 micron on whitefly scale, 30% Conidia 2.0×1.0 micron on thrip juveniles (nymphs), 10% Conidia 2.5×1.3 micron on thrip pupae. Underside of mycelium thallus sparsely creased, Mycelium thallus removes from the agar very easily.

L. muscarium strain K4V2 was isolated from whitefly in a cucumber greenhouse in Ruakaka, New Zealand. This L. muscarium isolate has been deposited in, the National Measurement Institute of Australia on 16 Mar. 2005 according to the Budapest Treaty for the purposes of patent procedure. The isolate has been accorded the deposit number NM05/44594.

K4V2 has the additional identifying characteristics—50% Conidia 2.0×1.5 μm, 30% Conidia 2.0×1.0 μm, 20% Conidia 1.0×1.0 μm, pathogenic to Whitefly adults, while Blastospores pathogenic to aphids. Underside of mycelium thallus frequently creased, Mycelium thallus difficult to remove from agar surface.

L. muscarium strain K4V4 was isolated from isolated from an outdoor organic tamarillo crop. This L. muscarium isolate has been deposited in the National Measurement Institute of Australia on 3 Mar. 2006 according to the Budapest Treaty for the purposes of patent procedure. The isolate has been accorded the deposit number NM06/00007.

K4V4 has the additional identifying characteristics—50% Conidia 1.0×0.5 μm, pathogenic to whitefly scale and adults, very aggressive at low humidity 65-75%, high temp 28-32°. Generally v.1>75%. 50% Condidia 0.5×0.5 μm. Underside of mycelium thallus sparsely creased, Mycelium thallus diffuses custard yellow to light orange pigment in media.

NMIA No. NM05/44593, NMIA No. NM05/44594, NMIA No. NM06/00007 and other suitable isolates of L. muscarium may be used in combination with one or more lipids or functional variants of the invention, and are particularly effective biological control agents, being capable of surviving interrupted wet periods, desiccation, and colonising, incapacitating and killing phytopathogenic insects such as, but not limited to, aphids, whitefly, mealy bugs, Varroa mite, and thrips, in the field.

Lecanicillium longisporum is an entomopathogenic fungi that is particularly pathogenic to aphids. Lecanicillium longisporum strain KT4L1 was isolated from aphids in Barley grass Banker plants in Franklin, Auckland, New Zealand. This L. longisporum isolate has been deposited in the National Measurement Institute of Australia on 3 Mar. 2006 according to the Budapest Treaty for the purposes of patent procedure. The isolate has been accorded the deposit number NM06/00009.

The isolate KT4L1 has the following identifying characteristics: 100% Condidia 6.0×2.1 μm, Mycelium thallus is offwhite to yellow growing very roughly which could be described as lumpy in consistency. Mycelium thallus diffuses light red brown colour into agar.

NMIA No. NM06/00009 and other suitable isolates of L. longisporum may be used in combination with one or more lipids, or functional variants of the invention, and are particularly effective biological control agents, being capable of surviving interrupted wet periods, desiccation, and colonising, incapacitating and killing phytopathogenic insects such as aphids, in the field.

Paecilomyces fumosoroseus is an entomopathogenic fungi found in infected and dead insects, and in some soils. P. fumosoroseus typically infects whiteflies, thrips, aphids, and caterpillars.

The K4P1 strain of Paecilomyces fumosoroseus was isolated from Diamond Back Moth caterpillar present on cabbage in Runciman, New Zealand. This P. fumosoroseus isolate has been deposited in the National Measurement Institute of Australia on 3 Mar. 2006 according to the Budapest Treaty for the purposes of patent procedure. The isolate has been accorded the deposit number NM06/00008.

NMIA No. NM06/00008 and other suitable isolates of P. fumosoroseus may be used in combination with one or more lipids or functional variants of the invention, and are particularly effective biological control agents, being capable of surviving interrupted wet periods, desiccation, and colonising, incapacitating and killing phytopathogenic insects such as, but not limited to, whitefly, Varroa mite, and Lepidoptera caterpillar in the field.

As discussed above, many insects have developed resistance to a number of insecticides; in these and other instances, compositions of the invention, optionally comprising or administered together with one or more fungal isolates such as those described above provide an effective alternative for insect control. This potent activity in the control of insects, such as those that cause human or plant disease, coupled with the absence of any observations of mammalian or plant pathogenicity induced by these agents demonstrate that lipids of the invention, and when present the fungal isolates of these species, have desirable attributes for use as a biological control agent.

The present invention provides a composition which comprises one or more lipids of the invention or functional variants thereof, together with one or more entomopathogenic fungi and at least one carrier.

The composition may include multiple strains of entomopathogenic fungi, and in certain embodiments, multiple strains may be utilised to target a number of phytopathogenic species, or a number of different developmental stages of a single phytopathogen, or indeed a combination of same. For example, the pupal form of a phytopathogenic insect may be targeted with one fungal, strain, while the adult form of the phytopathogenic insect may be targeted with another fungal strain, wherein both strains are included in a composition of the invention. In other embodiments, three strains or less will be preferred, and frequently a single strain will be preferred.

Suitably, the composition comprises fungi selected from the group consisting of Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

Particularly contemplated are compositions comprising one or more lipids of the invention or functional variants thereof and Lecanicillium muscarium strain K4V1 (NM05/44593) or a strain having the identifying characteristics thereof, compositions comprising one or more lipids of the invention or functional variants thereof and Lecanicillium muscarium strain K4V2 (NM05/44594) or a strain having the identifying characteristics thereof, and compositions comprising one or more lipids of the invention or functional variants thereof and both Lecanicillium muscarium strain K4V1 (NM05/44593) or a strain having the identifying characteristics thereof, compositions comprising one or more lipids of the invention or functional variants thereof and Lecanicillium muscarium strain K4V2 (NM05/44594) or a strain having the identifying characteristics thereof.

Examples of compositions comprising pesticidal or anti-phytopathogenic fungi are well known in the art, and include those described in, for example, PCT/US94/11542 (published as WO95/10597) to Mycotech Corporation, PCT/US2002/037218 (published as WO2003/043417) to The United States of America as represented by The Secretary of Agriculture, U.S. Pat. No. 4,530,834 to McCabe et al., U.S. patent application Ser. No. 10/657,982 (published as US 2004/0047841) to Wright et al., and PCT/NZ2009/000217 (published as WO2010/044680), each incorporated by reference herein in its entirety.

To be suitable for application to, for example, a plant or its surroundings, said at least one carrier is an agriculturally acceptable carrier, more preferably is selected from the group consisting of a filler stimulant, an anti-caking agent, a wetting agent, an emulsifier, and an antioxidant, more preferably said composition comprises at least one of each of a filler stimulant, an anti-caking agent, a wetting agent, an emulsifier, and an antioxidant. Preferably, said filler stimulant is a carbohydrate source, such as a disaccharide including, for example, sucrose, fructose, glucose, or dextrose, said anti-caking agent is selected from talc, silicon dioxide, calcium silicate, or kaelin clay, said wetting agent is skimmed milk powder, said emulsifier is a soy-based emulsifier such as lecithin or a vegetable-based emulsifier such as monodiglyceride, and said antioxidant is sodium glutamate or citric acid. However, other examples well known in the art may be substituted, provided the ability of the composition to support insecticidal efficacy, and fungal viability where necessary, is maintained.

Preferably, said composition is a biological control composition. The concentration of the insecticidal lipid of the invention present in the composition that is required to be effective as a biological control agent may vary depending on the end use, physiological condition of the plant; type (including insect species), concentration and degree of pathogen infection; temperature, season, humidity, stage in the growing season, the age of plant, number and type of conventional insecticides or other treatments (including fungicides) being applied; and physical treatments, such as plant treatments such as deleafing and pruning, may all be taken, into account in formulating the composition.

Insecticidal Compositions

Lipid Compositions and Methods of Use

The inventors contemplate that the lipid compositions disclosed herein will find particular utility as BCA compositions for topical and/or systemic application, for example to field crops, grasses, fruits and vegetables, lawns, trees, and/or ornamental plants. Alternatively, the lipids disclosed herein may be formulated as a spray, dust, powder, or other aqueous, atomized or aerosol for killing an insect, or controlling an insect population. The lipid compositions disclosed herein may be used prophylactically, or alternatively, may be administered to an environment once target insects have been identified in the particular environment to be treated. The lipid compositions may comprise an individual lipid or may contain various combinations of the lipids disclosed herein.

Regardless of the method of application, the amount of the active lipid component(s) is applied at an insecticidally-effective amount, which will vary depending on such factors as, for example, the specific target insects to be controlled, the specific environment, location, plant, crop, or agricultural site to be treated, the environmental conditions, and the method, rate, concentration, stability, and quantity of application of the insecticidally-active lipid composition. The formulations may also vary with respect to climatic conditions, environmental considerations, and/or frequency of application and/or severity of insect infestation.

The compositions described may be made by formulating the one or more lipids, functional variants or functional fragments thereof, optionally together with the fungal cell and/or spore suspension, with the desired agriculturally-acceptable carrier. The compositions may be formulated prior to administration in an appropriate means such as lyophilized, freeze-dried, desiccated, or in an aqueous carrier, medium or suitable diluent, such as saline or other buffer. The formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. The term, “agriculturally-acceptable carrier” covers all adjuvants, inert components, dispersants, surfactants, tackifiers, binders, etc. that are ordinarily used in insecticide formulation technology; these are well known to those skilled in insecticide formulation. The formulations may be mixed with one or more solid or liquid adjuvants and prepared by various means, e.g., by homogeneously mixing, blending and/or grinding the insecticidal composition with suitable adjuvants using conventional formulation techniques.

The compositions may include one or more fungal strains, may include one or more bacterial species, or both. Exemplary bacterial species include those such as B. thuringiensis, B. megaterium, B. subtilis, B. cereus, E. coli, Salmonella spp., Agrobacterium spp., or Pseudomonas spp.

Oil Flowable Suspensions

In one exemplary embodiment, the bioinsecticide composition comprises an oil flowable suspension of one or more lipids of the invention, functional variants or functional fragments thereof, optionally together with one or more fungal cells, including one or more fungal cells which expresses one or more of the novel proteins disclosed herein.

Water-Dispersible Granules

In another important exemplary embodiment, the bioinsecticide composition comprises a water dispersible granule. This granule comprises one or more lipids of the invention, functional variants or functional fragments thereof, optionally together with one or more fungal cells, including one or more fungal cells which produces a lipid of the invention disclosed herein.

Powders, Dusts, and Spore Formulations

In a third important exemplary embodiment, the bioinsecticide composition comprises a wettable powder, dust, spore formulation, cell pellet, or colloidal concentrate. This powder comprises one or more lipids of the invention, functional variants or functional fragments thereof, optionally together with one or more fungal cells, including one or more fungal cells which produces a lipid of the invention disclosed herein. Such dry forms of the insecticidal compositions may be formulated to dissolve immediately upon wetting, or alternatively, dissolve in a controlled-release, sustained-release, or other time-dependent manner. Such compositions may be applied to, or ingested by, the target insect, and as such, may be used to control the numbers of insects, or the spread of such insects in a given environment.

Aqueous Suspensions and Fungal Cell Filtrates or Lysates

In a fourth important exemplary embodiment, the bioinsecticide composition comprises an aqueous suspension of one or more lipids of the invention, functional variants or functional fragments thereof, optionally together with one or more fungal cells, including one or more fungal cells capable of producing a lipid of the invention disclosed herein. Such aqueous suspensions may be provided as a concentrated stock solution which is diluted prior to application, or alternatively, as a diluted solution ready-to-apply.

When the insecticidal compositions comprise intact cells producing the lipid of interest, such cells may be formulated in a variety of ways. They may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, suspensions, emulsifiable concentrates, or the like. The ingredients may include rheological agents, surfactants, emulsifiers, dispersants, or polymers.

Alternatively, the novel insecticidal lipids may be prepared by native or recombinant expression systems in vitro and isolated for subsequent field application. Such lipids may be either in crude cell lysates, suspensions, colloids, etc., or alternatively may be purified, refined, buffered, and/or further processed, before formulating in an active biocidal formulation. Likewise, under certain circumstances, it may be desirable to isolate lipids or spores from cultures producing the lipid and apply solutions, suspensions, or colloidal preparations of such lipids or spores as the active bioinsecticidal composition.

Multifunctional Formulations

In certain embodiments, for example those when the control of multiple insect species is desired, the insecticidal formulations described herein may also further comprise one or more chemical pesticides, (such as chemical pesticides, nematocides, fungicides, virucides, microbicides, amoebicides, insecticides, etc.), and/or one or more lipids having the same, or different insecticidal activities or insecticidal specificities, as the insecticidal lipids identified herein. The insecticidal lipids may also be used in conjunction with other treatments such as fertilizers, weed killers, cryoprotectants, surfactants, detergents, insecticidal soaps, dormant oils, polymers, and/or time-release or biodegradable carrier formulations that permit long-term dosing of a target area following a single application of the formulation. Likewise the formulations may be prepared into edible “baits” or fashioned into insect “traps” to permit feeding or ingestion by a target insect of the insecticidal formulation.

The insecticidal compositions of the invention may also be used in consecutive or simultaneous application to an environmental site singly or in combination with one or more additional insecticides, pesticides, chemicals, fertilizers, or other compounds.

Compositions Comprising Fungi

For use as a biological control agent, when present in the composition the entomopathogenic fungi of the invention should be in a reproductively viable form. The term reproductively viable as used herein includes mycelial and spore forms of the fungi. For example, for most purposes, fungal strains are desirably incorporated into the composition in the form of spores (conidia or blastospores). Spores are obtainable from all the fungal strains described herein, and may be produced using known art techniques. Compositions of the invention comprising one or more lipids of the invention, functional variants or functional fragments thereof, optionally together with one or more fungal cells, including one or more spores obtained from fungal strains described herein form a further aspect of the invention. The concentration of the fungal spores in the composition will depend on the utility to which the composition is to be put. An exemplary concentration range is from about 1×10⁶ to 1×10¹² spores per ml, preferably from about 1×10⁷ to 2×10¹⁰, and more preferably 1×10⁷ to 1×10⁸ spores per ml.

In theory one infective unit should be sufficient to infect a host but in actual situations a minimum number of infective units are required to initiate an infection. The concept of lethal dose (LD) regularly used with chemical pesticides is inappropriate for those microbial pesticides in which certain elements of entomopathogenic activity are reliant on colonisation of the plant or its surroundings by the entomopathogenic fungi. Concepts of infective dose (ID) or infective concentration (IC) are more precise or applicable. ID or IC refer to the actual number of infective units needed to initiate infection or the number of infective units exposed to the pathogen to cause death. Therefore, the number of infective units applied in the field or greenhouse against a pathogen will affect the degree of control. It is important to apply the desired concentration of the anti-phytopathogenic fungi, properly placed and at the right time, to obtain good control of the pest: this is known as the “infection threshold”.

It will be apparent that the concentration of fungal spores in a composition formulated for application may be less than that in a composition formulated for, for example, storage. The Applicants have determined that with the entomopathogenic fungi described herein, infection threshold occurs at about 10⁷ spores per ml of sprayable solution, when applied at a rate of about 1 L per hectare. Accordingly, in one example, a composition formulated for application will preferably have a concentration of at least about 10⁷ spores per ml. In another example, a composition formulated for storage (for example, a composition such as a wettable powder capable of formulation into a composition suitable for application) will preferably have a concentration of about 10¹⁰ spores per gram. It will be apparent that the spore concentration of a composition formulated for storage and subsequent formulation into a composition suitable for application must be adequate to allow said composition for application to also be sufficiently concentrated so as to be able to be applied to reach infection threshold.

In certain embodiments, the composition is a stable composition capable of supporting insecticidal efficacy (for example, of one or more lipids) for a period greater than about two weeks, preferably greater than about one month, about two months, about three months, about four months, about five months, more preferably greater than about six months. To be suitable for use in embodiments utilising one or more entomopathogens, such as an entomopathogenic fungi as described herein, the composition preferably is able to support reproductive viability of the entomopathogen or entomopathogenic efficacy for a period greater than about six months.

Using conventional solid substrate and liquid fermentation technologies well known in the art, the insecticidal lipids of the invention can be produced in sufficient amounts to allow use as biological control agents. For example, lipids and lipid fractions of the invention may be produced in sufficient quantity using these growing techniques, and exemplary techniques are presented herein in the Examples. Growth is generally effected under aerobic conditions at any temperature satisfactory for growth of the organism. For example, for B. bassiana, a temperature range of from 10 to 32° C., preferably 25 to 30° C., and most preferably 23° C., is preferred. The pH of the growth medium is slightly acid to neutral, that is, about 5.0 to 7.0, and most preferably 5.5. Incubation time is sufficient for the isolate to reach a stationary growth phase, about 21 days when incubated at 23° C., and will occur in normal photoperiod.

Spores from selected strains can be produced in bulk for field application using nutrient film, submerged culture, and rice substrate growing techniques. The spores may be harvested by methods well known in the art, for example, by conventional filtering or sedimentary methodologies (eg. centrifugation) or harvested dry using a cyclone system. Spores can be used immediately or stored, chilled at 0° to 6° C., preferably 2° C., for as long as they remain reproductively viable. It is however generally preferred that when not incorporated into a composition of the invention, use occurs within two weeks of harvesting.

Similarly, when required, the one or more lipids produced by B. bassiana K4B3 may be separated from the B. bassiana K4B3 by methods well known in the art, for example, by fractionation, filtering or sedimentary methodologies (eg. centrifugation), whether in combination with one or more cell-lysis steps (for example, for intracellular lipids) or not (for example, for lipids that are secreted into the growth media).

The composition of the invention may also include one or more carriers, preferably one or more agriculturally acceptable carriers. In one embodiment the carrier, such as an agriculturally acceptable carrier, can be solid or liquid. Carriers useful herein include any substance typically used to formulate agricultural composition.

In one embodiment the agriculturally acceptable carrier may be selected from the group comprising fillers, solvents, excipients, surfactants, suspending agents, speaders/stickers (adhesives), antifoaming agents, dispersants, wetting agents, drift reducing agents, auxiliaries, adjuvants or a mixture thereof.

Compositions of the invention may be formulated as, for example, concentrates, solutions, sprays, aerosols, immersion baths, dips, emulsions, wettable powders, soluble powders, suspension concentrates, dusts, granules, water dispersible granules, microcapsules, pastes, gels and other formulation types by well-established procedures.

These procedures include mixing and/or milling of the active ingredients with agriculturally acceptable carrier substances, such as fillers, solvents, excipients, surfactants, suspending agents, speaders/stickers (adhesives), antifoaming agents, dispersants, wetting agents, drift reducing agents, auxiliaries and adjuvants.

In one embodiment solid carriers include but are not limited to mineral earths such as silicic acids, silica gels, silicates, talc, kaolin, attapulgus clay, limestone, lime, chalk, bole, loess, clay, dolomite, diatomaceous earth, aluminas calcium sulfate, magnesium sulfate, magnesium oxide, ground plastics, fertilizers such as ammonium sulfate, ammonium phosphate, ammonium nitrate, and ureas, and vegetable products such as grain meals, bark meal, wood meal, and nutshell meal, cellulosic powders and the like. As solid carriers for granules the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

In one embodiment the carrier may also be liquid, for example, water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulfoxide, or N-methylpyrrolidone; liquefied gases; or the like or a mixture thereof.

In one embodiment surfactants include nonionic surfactants, anionic surfactants, cationic surfactants and/or amphoteric surfactants and promote the ability of aggregates, to remain in solution during spraying.

Spreaders/stickers promote the ability of the compositions of the invention to adhere to plant surfaces. Examples of surfactants, spreaders/stickers include but are not limited to Tween and Triton (Rhom and Hass Company), Deep Fried™, Fortune®, Pulse, C. Daxoil®, Codacide Oil®, D-C. Tate®, Supamet Oil, Bond®, Penetrant, Glowelt® and Freeway, Citowett®, Fortune Plus™, Fortune Plus Lite, Fruimec, Fruimec lite, alkali metal, alkaline earth metal and ammonium salts of aromatic sulfonic acids, e.g., ligninsulfonic acid, phenolsulfonic acid, naphthalenesulfonic acid and dibutylnaphthalenesulfonic acid, and of fatty acids, alkyl and alkylaryl sulfonates, and alkyl, lauryl ether and fatty alcohol sulfates, and salts of sulfated hexadecanols, heptadecanols, and octadecanols, salts of fatty alcohol glycol ethers, condensation products of sulfonated naphthalene and naphthalene derivatives with formaldehyde, condensation products of naphthalene or naphthalenesulfonic acids with phenol and formaldehyde, polyoxyethylene octylphenol ethers, ethoxylated isooctylphenol, ethoxylated octylphenol and ethoxylated nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methyl cellulose. Where selected for inclusion, one or more agricultural surfactants, such as Tween are desirably included in the composition according to known protocols.

Wetting agents reduce surface tension of water in the composition and thus increase the surface area over which a given amount of the composition may be applied. Examples of wetting agents include but are not limited to salts of polyacrylic acids, salts of lignosulfonic acids, salts of phenolsulfonic or naphthalenesulfonic acids, polycondensates of ethylene oxide with fatty alcohols or fatty acids or fatty esters or fatty amines, substituted phenols (particularly alkylphenols or arylphenols), salts of sulfosuccinic acid esters, taurine derivatives (particularly alkyltaurates), phosphoric esters of alcohols or of polycondensates of ethylene oxide with phenols, esters of fatty acids with polyols, or sulfate, sulfonate or phosphate functional derivatives of the above compounds.

In one embodiment the preferred method of applying the compound or composition of the invention is to spray a dilute or concentrated solution by handgun or commercial airblast.

As described above, the compositions of the present invention may be used alone or in combination with one or more other agricultural agents, including pesticides, insecticides, acaracides, fungicides or bactericides (provided such fungicides or bactericides are not detrimental or toxic to any fungi or bacteria present in the composition), herbicides, antibiotics, antimicrobials, nemacides, rodenticides, entomopathogens, pheromones, attractants, plant growth regulators, plant hormones, insect growth regulators, chemosterilants, microbial pest control agents, repellents, viruses, phagostimulents, plant nutrients, plant fertilisers and biological controls. When used in combination with other agricultural agents the administration of the two agents may be separate, simultaneous or sequential. Specific examples of these agricultural agents are known to those skilled in the art, and many are readily commercially available.

Examples of plant nutrients include but are not limited to nitrogen, magnesium, calcium, boron, potassium, copper, iron, phosphorus, manganese, molybdenum, cobalt, boron, copper, silicon, selenium, nickel, aluminum, chromium and zinc.

Examples of antibiotics include but are not limited to oxytetracyline and streptomycin.

Examples of fungicides include but are not limited to the following classes of fungicides: carboxamides, benzimidazoles, triazoles, hydroxypyridines, dicarboxamides, phenylamides, thiadiazoles, carbamates, cyano-oximes, cinnamic acid derivatives, morpholines, imidazoles, beta-methoxy acrylates and pyridines/pyrimidines.

Further examples of fungicides include but are not limited to natural fungicides, organic fungicides, sulphur-based fungicides, copper/calcium fungicides and elicitors of plant host defences.

Examples of natural fungicides include but are not limited to whole milk, whey, fatty acids or esterified fatty acids.

Examples of organic fungicides include but are not limited to any fungicide which passes an organic certification standard such as biocontrol agents, natural products, elicitors (some of may also be classed as natural products), and sulphur and copper fungicides (limited to restricted use).

An example of a sulphur-based fungicide is Kumulus™ DF (BASF, Germany).

An example of a copper fungicide is Kocide® 2000 DF (Griffin Corporation, USA).

Examples of elicitors include but are not limited to chitosan, Bion™, BABA (DL-3-amino-n-butanoic acid, β-aminobutyric acid) and Milsana™ (Western Farm Service, Inc., USA).

In some embodiments non-organic fungicides may be employed. Examples of non-organic fungicides include but are not limited to Bravo™ (for control of PM on cucurbits); Supershield™ (Yates, NZ) (for control of Botrytis and PM on roses); Topas® 200EW (for control of PM on grapes and cucurbits); Flint™ (for control of PM on apples and cucurbits); Amistar® WG (for control of rust and PM on cereals); and Captan™, Dithane™, Euparen™, Rovral™, Scala™, Shirlan™, Switch™ and Teldor™ (for control of Botrytis on grapes).

Examples of pesticides include but are not limited to azoxystrobin, bitertanol, carboxin, Cu₂O, cymoxanil, cyproconazole, cyprodinil, dichlofluamid, difenoconazole, diniconazole, epoxiconazole, fenpiclonil, fludioxonil, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, metconazole, oxadixyl, pefurazoate, penconazole, pencycuron, prochloraz, propiconazole, pyroquilone, SSF-109, spiroxamin, tebuconazole, thiabendazole, tolifluamid, triazoxide, triadimefon, triadimenol, triflumizole, triticonazole and uniconazole.

An example of a biological control agent other than a fungal strain described herein is the BotryZen™ biological control agent comprising Ulocladium oudemansii.

The compositions may also comprise a broad range of additives such as stablisers and penetrants used to enhance the active ingredients and so-called ‘stressing’ additives to improve spore vigor, germination and survivability such as potassium chloride, glycerol, sodium chloride and glucose. Additives may also include compositions which assist in maintaining microorganism viability in long term storage, for example unrefined corn oil and so called invert emulsions containing a mixture of oils and waxes on the outside and water, sodium alginate and conidia on the inside.

As will be appreciated by those skilled in the art, it is important that any additives used are present in amounts that do not interfere with the effectiveness of the biological control agents.

Examples of suitable compositions including carriers, preservations, surfactants and wetting agents, spreaders, and nutrients are provided in U.S. Pat. No. 5,780,023, incorporated herein in its entirety by reference.

The Applicants have also determined that many commonly used fungicides do not adversely affect the entomopathogenic fungi, when present, described herein. The compositions of the invention comprising said fungi may therefore also include such fungicides. Alternatively, the compositions may be used separately but in conjunction with such fungicides in control programmes.

The invention also provides a method of producing a composition comprising one or more lipids of the invention or one or more functional variants thereof and one or more entomopathogenic fungi described herein, said method comprising providing a reproductively viable form of said entomopathogenic fungi, and combining said reproductively viable form of said entomopathogenic fungi with one or more lipids of the invention or functional variants thereof and at least one agriculturally acceptable carrier.

The compositions may be prepared in a number of forms. One preparation comprises a powdered form of a composition of the invention which may be dusted on to a plant or its surroundings. In a further form, the composition is mixed with a diluent such as water to form a spray, foam, gel or dip and applied appropriately using known protocols. In a presently preferred embodiment, a composition formulated as described above is mixed with water using a pressurised sprayer at about 1 gm/L, or about 1 to 3 kg/ha in no less than 1000 L water per ha. Preferably, Deep Fried™ or Fortune Plus™ is added to the composition as a UV and desiccation protection agent at about 1 ml/L. Compositions comprising L. muscarium, L. longisporum, or P. fumosoroseus can be applied in a similar manner.

Compositions formulated for other methods of application such as injection, rubbing or brushing, may also be used, as indeed may any known art method. Indirect applications of the composition to the plant surroundings or environment such as soil, water, or as seed coatings are potentially possible.

As discussed above, the concentration at which the compositions of the invention, including those comprising entomopathogenic fungi as described herein are to be applied so as to be effective biological control agents may vary depending on the end use, physiological condition of the plant; type (including insect species), concentration and degree of pathogen infection; temperature, season, humidity, stage in the growing season and the age of plant; number and type of conventional insecticides or other treatments (including fungicides) being applied; and plant treatments (such as leaf plucking and pruning).

Other application techniques, including dusting, sprinkling, soil soaking, soil injection, seed coating, seedling coating, foliar spraying, aerating, misting, atomizing, fumigating, aerosolizing, and the like, are also feasible and may be required under certain circumstances such as e.g., insects that cause root or stalk infestation, or for application to delicate vegetation or ornamental plants. These application procedures are also well-known to those of skill in the art.

The insecticidal compositions of the present invention may also be formulated for preventative or prophylactic application to an area, and may in certain circumstances be applied to pets, livestock, animal bedding, or in and around farm equipment, barns, domiciles, or agricultural or industrial facilities, and the like.

The concentration of insecticidal composition which is used for environmental, systemic, topical, or foliar application will vary widely depending upon the nature of the particular formulation, means of application, environmental conditions, and degree of biocidal activity. Typically, the bioinsecticidal composition will be present in the applied formulation at a concentration of at least about 1% by weight and may be up to and including about 99% by weight. Dry formulations of the lipid compositions may be from about 0.01% to about 99% or more by weight of the lipid composition, while liquid formulations may generally comprise from about 1% to about 99% or more of the active ingredient by weight. As such, a variety of formulations are preparable, including those formulations that comprise from about 5% to about 95% or more by weight of the insecticidal lipid, including those formulations that comprise from about 10% to about 90% or more by weight of the insecticidal lipid. Naturally, compositions comprising from about 15% to about 85% or more by weight of the insecticidal lipid, and formulations comprising from about 20% to about 80% or more by weight of the insecticidal lipid are also considered to fall within the scope of the present disclosure.

In the case of compositions in which intact fungal cells that contain the insecticidal lipid are included, preparations will generally contain from about 10⁴ to about 10⁸ cells/mg, although in certain embodiments it may be desirable to utilize formulations comprising from about 10² to about 10⁴ cells/mg, or when more concentrated formulations are desired, compositions comprising from about 10⁸ to about 10¹⁰ or 10¹¹ cells/mg may also be formulated. Alternatively, cell pastes, spore concentrates, or lipid suspension concentrates may be prepared that contain the equivalent of from about 10¹² to 10¹³ cells/mg of the active lipid, and such concentrates may be diluted prior to application.

The insecticidal formulation described above may be administered to a particular plant or target area in one or more applications as needed, with a typical field application rate per hectare ranging on the order of from about 50 g/hectare to about 500 g/hectare of active ingredient, or alternatively, from about 500 g/hectare to about 1000 g/hectare may be utilized. In certain instances, it may even be desirable to apply the insecticidal formulation to a target area at an application rate of from about 1000 g/hectare to about 5000 g/hectare or more of active ingredient. In fact, all application rates in the range of from about 50 g of active lipid per hectare to about 10,000 g/hectare are contemplated to be useful in the management, control, and killing, of target insect pests using such insecticidal formulations. As such, rates of about 100 g/hectare, about 200 g/hectare, about 300 g/hectare, about 400 g/hectare, about 500 g/hectare, about 600 g/hectare, about 700 g/hectare, about 800 g/hectare, about 900 g/hectare, about 1 kg/hectare, about 1.1 kg/hectare, about 1.2 kg/hectare, about 1.3 kg/hectare, about 1.4 kg/hectare, about 1.5 kg/hectare, about 1.6 kg/hectare, about 1.7 kg/hectare, about 1.8 kg/hectare, about 1.9 kg/hectare, about 2.0 kg/hectare, about 2.5 kg/hectare, about 3.0 kg/hectare, about 3.5 kg/hectare, about 4.0 kg/hectare, about 4.5 kg/hectare, about 6.0 kg/hectare, about 7.0 kg/hectare, about 8.0 kg/hectare, about 8.5 kg/hectare, about 9.0 kg/hectare, and even up to and including about 10.0 kg/hectare or greater of active lipid may be utilized in certain agricultural, industrial, and domestic applications of the pesticidal formulations described hereinabove.

For example, in certain applications, a composition comprising a fungus as described herein may be applied at a rate of from about 1×10¹⁰ to about 1×10¹⁵ spores per hectare, preferably from about 1×10¹² to about 1×10¹⁴ spores per hectare, more preferably from about 5×10¹² to about 1×10¹⁴ spores per hectare, more preferably about 1-3×10¹³ spores per hectare.

Representative application rates for liquid compositions include application rates in the range of from about 50 mL of active lipid per hectare to about 20 L/hectare are contemplated to be useful in the management, control, and killing, of target insect pests using such insecticidal formulations. As such, rates of about 100 mL/hectare, about 200 mL/hectare, about 300 mL/hectare, about 400 mL/hectare, about 500 mL/hectare, about 600 mL/hectare, about 700 mL/hectare, about 800 mL/hectare, about 900 mL/hectare, about 1 L/hectare, about 1.1 L/hectare, about 1.2 L/hectare, about 1.3 L/hectare, about 1.4 L/hectare, about 1.5 L/hectare, about 1.6 L/hectare, about 1.7 L/hectare, about 1.8 L/hectare, about 1.9 L/hectare, about 2.0 L/hectare, about 2.5 L/hectare, about 3.0 L/hectare, about 3.5 L/hectare, about 4.0 L/hectare, about 4.5 L/hectare, about 6.0 L/hectare, about 7.0 L/hectare, about 8.0 L/hectare, about 8.5 L/hectare, about 9.0 L/hectare, and even up to and including about 10.0 L/hectare or greater of active lipid may be utilized.

In a further aspect the present invention provides a method for controlling one or more phytopathogenic insects, the method comprising applying to a plant or its surroundings a lipid as described herein.

In one embodiment, the application is of one or more lipid fractions as exemplified herein together with one or more other entomopathogenic fungi as described herein.

Preferably, said one or more other fungi is selected from the group consisting of B. bassiana strain K4B3 (NMIA Accession No. V08/025855) or a strain having the identifying characteristics thereof; Lecanicillium muscarium strain K4V1 (NMIA Accession No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain. K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

Again, multiple lipids of the invention, whether or not in combination with multiple strains of the entomopathogenic fungi with activity against one or more phytopathogenic insect species, may be employed in the control process.

Young seedlings are typically most susceptible to damage from insect pests. Therefore, application of the compositions of the invention to freshly planted out crops, prior to emergence, is contemplated, as is application on emergence.

Repeated applications at the same or different times in a crop cycle are also contemplated. The insecticidal lipids of the invention, compositions comprising the insecticidal lipids of the invention may be applied either earlier or later in the season. This may be over flowering or during fruiting. The insecticidal lipids of the invention or compositions comprising the insecticidal lipids of the invention may also be applied immediately prior to harvest, or after harvest to rapidly colonise necrotic or senescing leaves, fruit, stems, machine harvested stalks and the like to prevent insect colonisation. The insecticidal lipids of the invention or compositions of the invention may also be applied to dormant plants in winter to slow insect growth on dormant tissues.

Application may be at a time before or after bud burst and before and after harvest. However, treatment preferably occurs between flowering and harvest. To increase efficacy, multiple applications (for example, 2 to 6 applications over the stages of flowering through fruiting) of the insecticidal lipids of the invention or a composition of the invention is preferred.

Reapplication of the insecticidal lipids of the invention or composition should also be considered after rain. Using insect infectivity prediction models or infection analysis data, application of the BCA can also be timed to account for insect infection risk periods.

In various exemplary embodiments, the lipids of the present invention and compositions comprising such lipids are not deleterious to the plants or plant surroundings to which they are applied at dosage rates capable of achieving insecticidal efficacy.

In the presently preferred embodiments, the insecticidal lipids of the invention or a composition comprising same is applied in a solution, for example as described above, using a pressurised sprayer. The plant parts should be lightly sprayed until just before run off, ideally to ensure thorough coverage. Applications may be made to any part of the plant and/or its surroundings, for example to the whole plant canopy, to the area in the canopy where the flowers and developing fruit are concentrated, or to the plant stem and/or soil, water or growth media adjacent to or surrounding the roots, tubers or the like.

Preferably the composition is stable. As used herein, the term “stable” refers to a composition capable of supporting insecticidal efficacy for several weeks, preferably about one, about two, about three, about four, preferably about five, more preferably about six months, or longer. Preferably, the composition is stable without a requirement for storage under special conditions, such as, for example, refrigeration or freezing.

The applied compositions control phytopathogenic insects. Phytopathogenic insects are responsible for many of the pre- and post-harvest diseases which attack plant parts and reduce growth rate, flowering, fruiting, production and may cause death of afflicted plants. As used herein, phytopathogenic insects include insects which are themselves plant pathogens, and insects which may act as a vector for other plant pathogens, for example, phytopathogenic fungi and bacteria. It will be appreciated that by controlling host insects which act as vectors for other phytopathogens, the incidence and/or severity of plant disease can be minimised.

Examples of the major phytopathogenic insects afflicting a number of important horticultural crops grown in New Zealand are presented in Table 1 below.

TABLE 1
Major Insect Pests

| | No. of | Planted | |
|---|---|---|---|
| Crop | Growers | area (ha) | Major Pest |
| Cherries | | 550 | Aphids |
| Potatoes | 321 | 10,611 | Aphids, whitefly |
| Tomatoes (indoor) | 390 | 167 | Whitefly, caterpillars |
| Brassicas | 227 | 3,746 | Whitefly, caterpillars |
| Squash | 181 | 6,560 | Whitefly, aphids |
| Tamarillos | 175 | 270 | Whitefly, aphids |
| Strawberries | 125 | 361 | Aphids, thrips |
| Cucumber (indoor) | | 55 | Aphids, thrips, whitefly |
| Onions | 150 | 5,488 | Thrips |
| Tomatoes (outdoor) | 80 | 609 | Whitefly, caterpillars, thrips |
| Capsicum | 142 | 87 | Thrips, aphids, whitefly, |
| | | | caterpillars |
| Lettuce | 252 | 1,287 | Aphids, thrips |
| Pumpkin | 125 | 1,033 | Whitefly, aphids |

Control of Hemiptera, such as whitefly, thrips, aphids, and caterpillars including in the crops outlined above using the lipids, the compositions, and the methods of the present invention is particularly contemplated. Control of Varroa mite using lipids of the invention, either alone or together with one or more B. bassiana fungal strains, or with L. muscarium, or Paecilomyces fumosoroseus and compositions of the present invention comprising same are also particularly contemplated.

The methods of the invention have particular application to plants and plant products, either pre- or post-harvest. For example, the composition of the invention may be applied to stored products of the type listed above including fruits, vegetables, cut flowers and seeds. Suitable application techniques encompass those identified above, particularly spraying.

The composition can potentially be used to treat or pretreat soils or seeds, as opposed to direct application to a plant. The composition may find use in plant processing materials such as protective coatings, boxes and wrappers.

Also encompassed by the present invention are plants, plant products, soils and seeds treated directly with a lipid of the invention or a composition of the invention.

The invention consists in the foregoing and also envisages constructions of which the following gives examples only and in no way limit the scope thereof.

### Example 1

### Bioassays of Insect Control

This example describes the development of a robust bioassay to determine the insecticidal efficacy of various lipids.

The target insect assay was developed and assessed using the criteria 1) availability, 2) susceptibility and 3) ease of use.

The target insect Myzus persicae (green peach aphid, order Hemiptera), reared on cabbage plants in a constant temperature room, was used in all experiments. To inoculate aphids with lipid fraction samples, aphids were transferred to a piece of cabbage leaf on the surface of a 1% water agar plate using 0.05% Tween 80 as a wetting agent between leaf and agar. Aphids of mixed age were used, usually between 30-50/Petri dish.

A hand-held Paasche airbrush was modified to take micro volumes and used to atomise 300 μl of test or control solutions. Subsequently, plates with treated aphids were maintained at 20° C., 12 h light:12 h dark and checked daily. Dead were removed. Counts of aphids inoculated were made directly after spraying to avoid including newborn aphids in % mortality, but no effort was made to remove neonate nymphs during incubation.

### Example 2

### Bioassays of Insect Control

This example describes the development of an expanded range of bioassays to determine the insecticidal efficacy.

Several target insect assays were developed and assessed using the criteria 1) availability, 2) susceptibility and 3) ease of use. Four insect systems were tested: whiteflies, diamond back moth, mealworm, and mosquito.

### 1. White Lies Nymphs (Hemiptera)

Whitefly is a major target species, currently lacking suitable control agents. Whitefly nymphs were obtained from Bioforce Ltd (Auckland, New Zealand). Samples of K4B3 lipid fractions and control broth are used to inoculate groups of approximately 100 nymphs through a Potter Tower.

2. Mealworm (Tenebrio molitor) larvae (Coleoptera)

Tenebrio larvae are obtained from biosuppliers. Ten larvae are sprayed with K4B3 lipid sample or water control and monitored for 2 weeks.

3. Diamondback Moth-DBM (Plutella xylostella) Larvae (Lepidoptera)

Diamondback moth is a major pest of brassica crops around the world and an insect which has become resistant to most control chemicals. A culture is obtained from Lincoln University for testing with the K4B3 lipid samples.

The standard method uses a mini-version of the Potter Tower. A hand-held A320 airbrush is modified to take, micro volumes to atomise lipid samples or control solutions. Larvae are maintained on small cabbage leaves held on the surface of a water agar plate (water+1% agar) using 0.05% Tween 80. Between 5-20 larvae of all sizes (1^{st} to prepupal) are used in each experiment. In one test, larvae are also dipped in drops of solution. Droplet feeding is attempted to determine if toxicity is topical or ingestion.

Thirty-eight 2^{nd}-6^{th} instar larvae are inoculated in the K4B3 lipid fractions and 19 in the control. Larvae are maintained at 20° C. in 16 hL:8 hD after inoculation.

### 4. Mosquito Larvae

Bioassays are conducted in which varying amounts of K4B3 lipid fractions are added to bottles of approximately 12.5 ml of water containing larvae of Culex perviligans. Larvae are nearing pupation and some may pupate during the experiment.

### Example 3

### Identification of Insecticidal Lipids

### Introduction

This example describes the identification of insecticidal lipids prepared from Beauveria bassiana K4B3.

### Methods

Cultures of Beauveria bassiana K4B3 were grown in shaken flasks and in a 200 L biofermentor. The freeze dried samples were extracted with methanol and dried down. The residue was dissolved in 50% ethanol and extracted with dichloromethane. The dichloromethane was washed with water to remove undesired co-extractives. A small portion of the remaining dichloromethane extract was dried down, redissolved in methanol and subjected to MS analysis.

Fractionation was performed over Strata SI-1 Silica columns (Phenomenex). The crude sample was dissolved in a small volume of dichloromethane, loaded onto the Silica cartridge and eluted with mixtures of isopropanol and dichloromethane (100% dichloromethane to 100% isopropanol in several steps), followed by mixtures of methanol and dichloromethane (100% dichloromethane to 100% methanol in several steps).

A Waters Acquity UPLC (Waters, Milford, Mass.) was coupled to a Waters-Micromass Quattro Premier triple quadropole spectrometer (Waters-Micromass, Manchester, UK). Separation was achieved using a Acquity C18 BEH 1.7 μm column 50×1 mm at 40° C. The column was eluted at 0.25 mL/min with water (A) and acetonitrile (B) mobile phase, each containing 0.1% formic acid. The initial composition was 100% A for 1 min followed by a linear gradient to 100% B between 1 and 7 minutes, then kept at 100% B between 7 and 9 min and finally the column was re-equilibrated with 100% A from 9 to 11 minutes.

The electrospray ionisation source was operated in positive mode at 100° C., capillary 3 kV, cone 60 V, nitrogen desolvation 200 l/h (250° C.,) cone gas 50 l/h, the collision cell was operated with 3 mbar argon. Daughter ion spectra from the protonated molecular cations were acquired using a collision energy (CE) of 40 eV. For quantitative analysis (not absolute, but relative to a reference sample) Selective Ion Recording (SIR) and multiple reaction monitoring (MRM) were used. Peak areas were compared to those obtained for a reference sample.

### Results

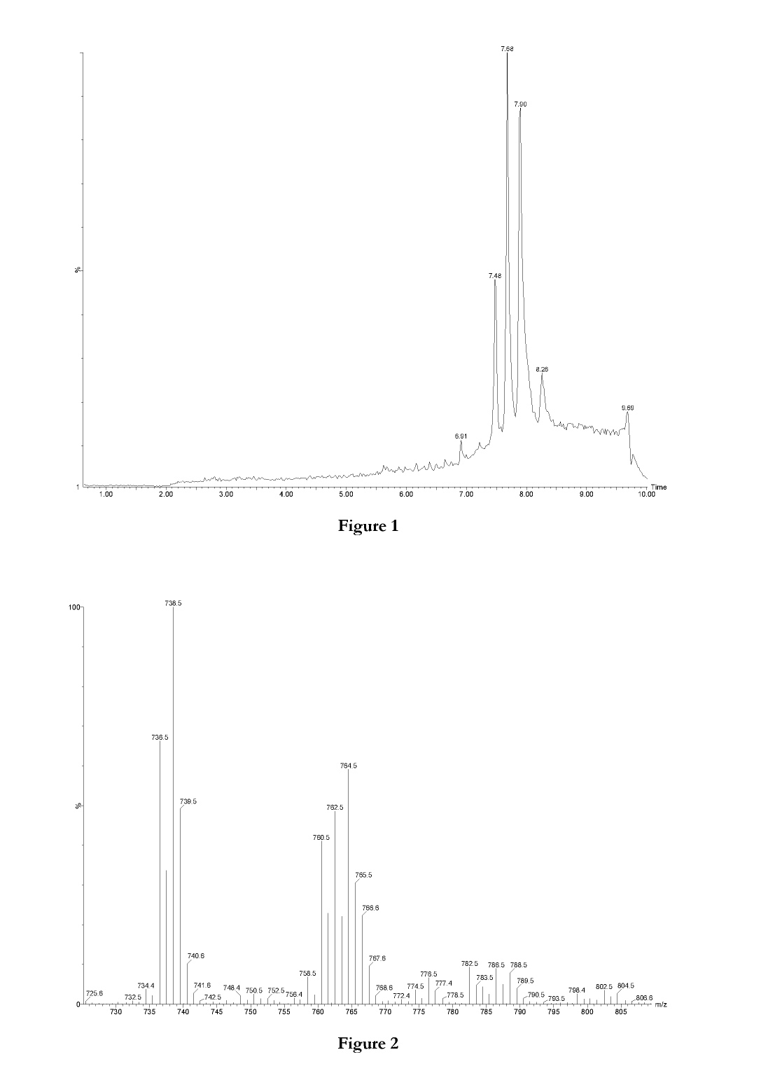

During the first methanol/dichloromethane fractionation step, the betaine lipids eluted from the silica column at a mixture of 20-40% methanol and 60-80% dichloromethane. Chromatography of organic solvent extracted fractions is shown in FIG. 1, where peaks comprising the exemplary lipids of interest can be seen.

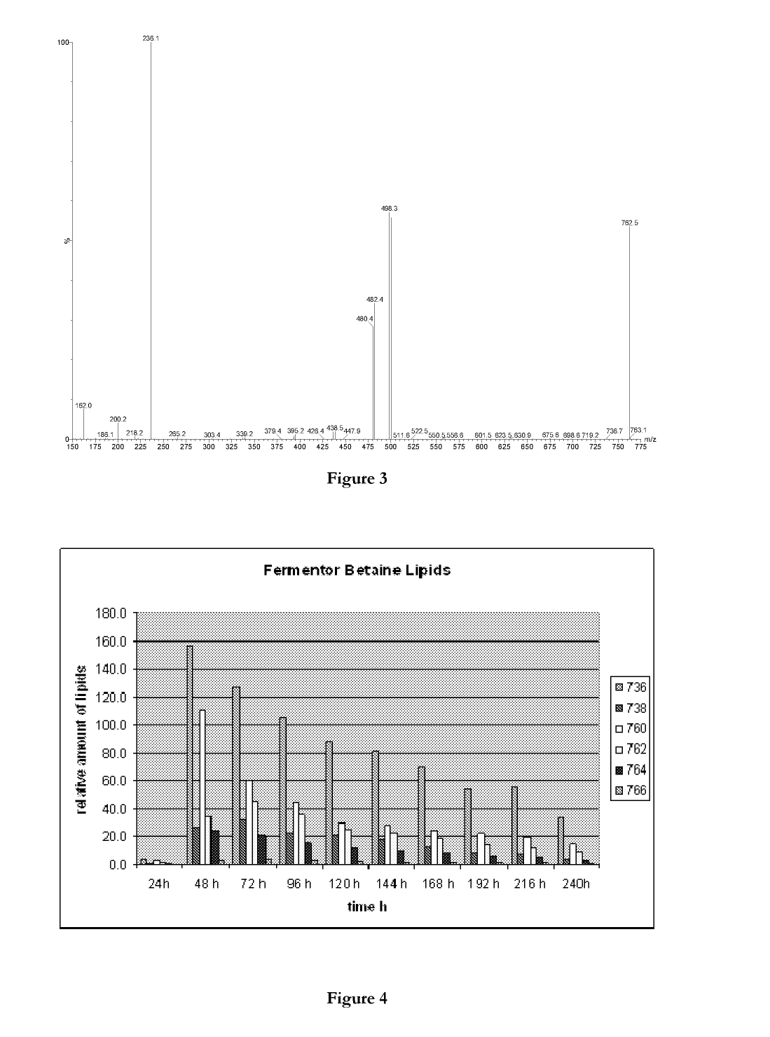

Fractions of interest were analysed using mass spectrometry (MS). Peaks of interest were selected for MS/MS (fragmentation analysis). As can be seen in FIG. 2, six lipids with m/z of 736, 738, 760, 762, 764, and 766, were observed.

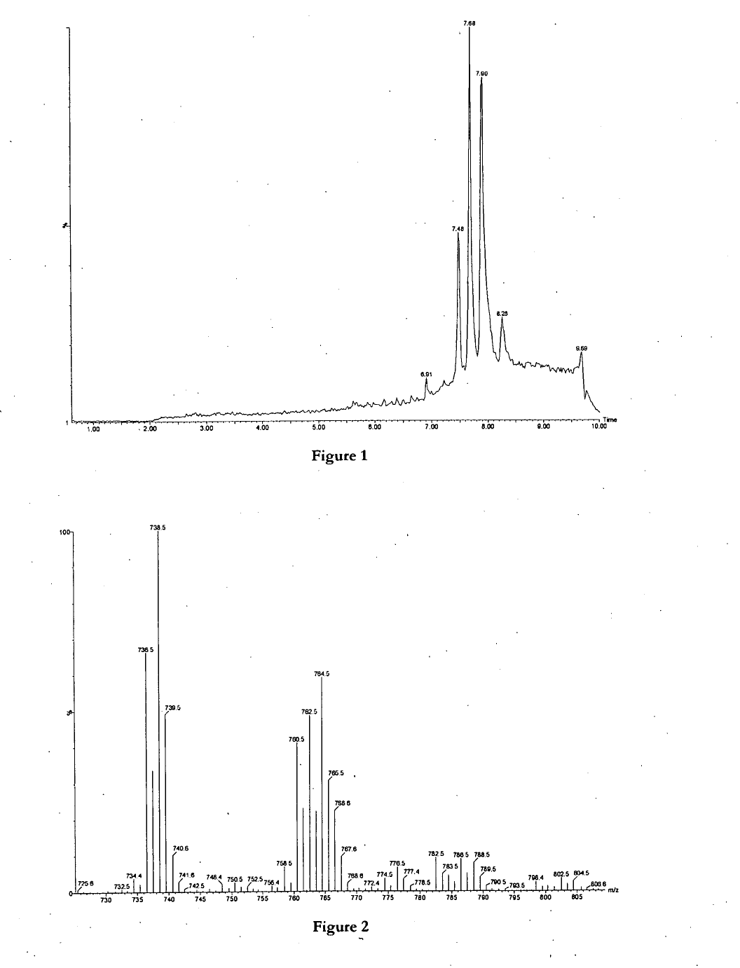

Further analysis was performed to resolve the structure of each lipid. A representative daughter spectrum of exemplary lipid having m/z of 762.5 is shown in FIG. 3. Here, the loss of the acyl part of the C18:2 fatty acid can be seen at m/z 500.3, and loss of the acyl part of C18:1 fatty acid can be seen at m/z 498.3. Subsequent losses of water molecules are evident at m/z 482.2 and 480.4. The common glyceryl-N,N,N-trimethylhomoserine fragment formed after loss of both fatty acids is shown at m/z 236.1. The peak observed at m/z 162.0 was formed after the subsequent loss of glycerol resulted in N,N,N-trimethylhomoserine.

Equivalent analyses of the five remaining exemplary lipids was also performed, such that the exemplary insecticidal lipids were determined by this analysis to have the general structure of Formula II as shown below

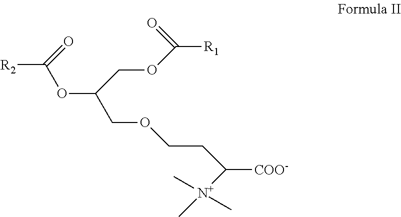

wherein R₁ and R₂ are each the aliphatic portion of a fatty acid.

The fatty acid composition of specific exemplary betaine lipids determined in this example is shown below in Table 2.

TABLE 2
Fatty acids in Betaine lipids

| M + H | fatty acid A | fatty acid B |
|---|---|---|
| 736 | C16:0 | C18:2 |
| 738 | C16:0 | C18:1 |
| 760 | C18:2 | C18:2 |
| 762 | C18:2 | C18:1 |
| 764A | C18:2 | C18:0 |
| 764B | C18:1 | C18:1 |
| 766 | C18:1 | C18:0 |

Evidence for the presence of both 764A and 764B was obtained from fragmentation data. M+H 764 shows an initial loss of C18:0, C18:1 as well as C18:2 fatty acids from the 764 parent lipid.

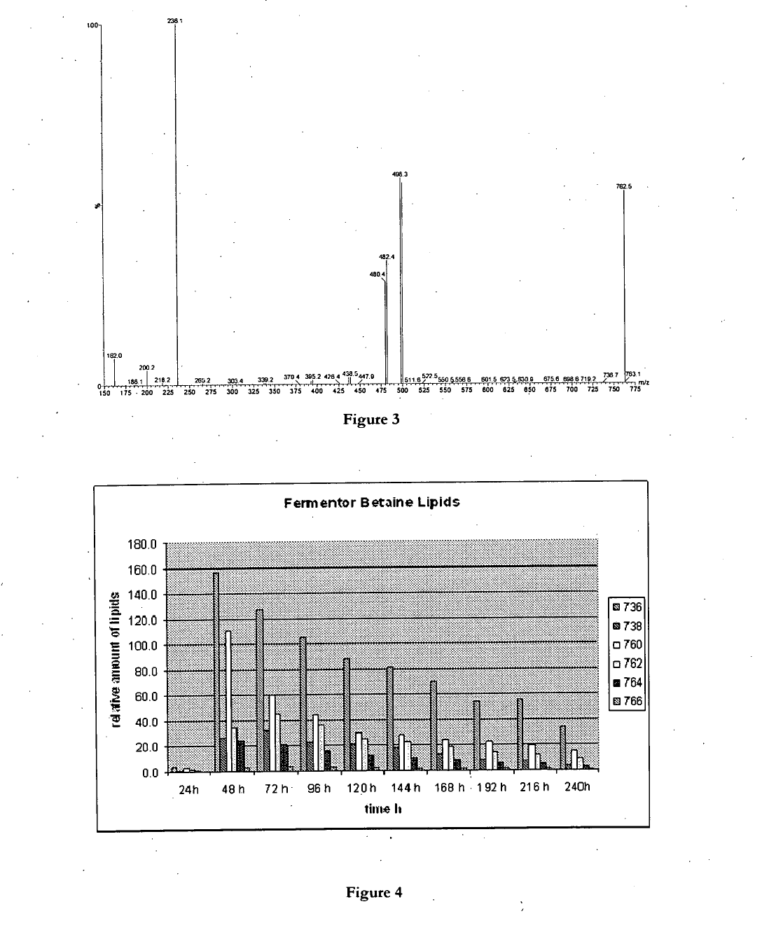

The relative amounts of the various betaine lipids produced at different timepoints during growth in the biofermentor is shown in FIG. 4. As can be seen, by 48 hrs growth lipid 760 is the predominant betaine lipid, and 48 hrs also represents the peak betaine lipid production of the time points tested.

### Discussion

This example elucidates the structure of exemplary insecticidal lipids from Beauveria bassiana K4B3, and provides a new class of biological control compounds.

### Example 4

### Identification of Insecticidal Lipids

### Introduction

This example describes the identification of insecticidal lipids prepared from Beauveria bassiana K4B3.

### Methods

Cultures of Beauveria bassiana K4B3 were grown in shaken flasks and in a 200 L biofermentor. The freeze dried samples were extracted with methanol and dried down.

Dried methanol extract (81.936 g) was redissolved in water (450 ml) and methanol (1 L), with the aid of sonication. Chloroform (500 ml) was added to the mixture, to give a single phase solution that was split into two equal 970 ml portions. Each portion was separately shaken with additional chloroform (250 ml) and water (225 ml), after which they separated into organic and aqueous phases. The combined aqueous phase (1820 ml) was split into two equal portions and each was then shaken with additional chloroform (250 ml). The resultant organic layers were combined with the bulk organic layer (990 ml), while the two portions of aqueous layer were recombined and were concentrated on a Buchi rotary evaporator before being freeze dried. The dried aqueous layer (74.764 g, 91.2% yield) was then stored in a −20° C. freezer.

The solvent was removed under vacuum from the combined organic phase to provide 2.728 g (3.3% yield) of chloroform extract, which was redissolved in 50 ml chloroform and stored in a −20° C. freezer.

DEAE-Sephadex (22.5 g) was first equilibrated with chloroform-methanol-0.8 M sodium acetate (30:60:8), and then washed with chloroform-methanol-water (30:60:8). Beaublast™ chloroform extract (2.706 g) was loaded onto the DEAE-Sephadex column in chloroform-methanol-water (30:60:8) and the non-retained (non-acidic fraction) was eluted with the same solvent. The acidic fraction was then eluted with chloroform-methanol-0.8 M sodium acetate (30:60:8).

Each fraction was separately concentrated to a small volume and was shaken with chloroform and 0.88% potassium chloride solution. Following separation, the lower organic phase from each fraction was dried over anhydrous magnesium sulphate before being filtered and concentrated to dryness under vacuum. The yield of non-acidic fraction was 1.308 g (48.4%) and of acidic fraction was 0.072 g (2.7%). Each fraction was dissolved in chloroform and was stored in a −20° C. freezer.

Normal phase chromatography of the non-acidic fraction (1.29 g) was performed on a silica gel 60 (BDH, 120 mesh, 121.5 g) column self-packed in chloroform. The sample was loaded in chloroform, and then eluted with chloroform, chloroform-acetone (9:1) and finally methanol (−7.8 bed volumes of each). Following evaporation of each of the sub-fractions to dryness under vacuum, the yields of the three sub-fractions were: chloroform sub-fraction, 0.609 g (47.2%); chloroform-acetone sub-fraction, 0.142 g (11.0%); and methanol sub-fraction, 0.508 g (39.4%). Each sub-fraction was dissolved in chloroform and stored in a −20° C. freezer.

Nanospray mass spectrometry (MS) was performed on a QStar Pulsar i Q-TOF mass spectrometer. Samples were diluted 1:20 with 1:1 chloroform-methanol containing 10 mM ammonium acetate prior to analysis.

### Results

Nanospray MS/MS (fragmentation analysis) was performed to resolve the structure of compounds present in the methanol sub-fraction. Seven betaine lipids with m/z of 736.6, 738.6, 758.6 760.6, 762.6, and 764.6, were observed.

The fatty acid composition of these betaine lipids is shown below in Table 3.

TABLE 3
Fatty acids in Betaine lipids

| m/z | fatty acid A | fatty acid B |
|---|---|---|
| 736.6 | C16:0 | C18:2 |
| 738.6 | C16:0 | C18:1 |
| 758.6 | C18:2 | C18:3 |
| 760.6 | C18:0 | C18:2 |
| 762.6 | C18:0 | C18:2 |
| 764.6 | C18:0 | C18:2 |
| 764.6 | C18:1 | C18:1 |

### Discussion

This example elucidates the Structure of exemplary insecticidal lipids from Beauveria bassiana K4B3, and provides a new class of biological control compounds.

### Example 5

### Bioassay of Insecticidal Lipids

### Introduction

This example describes the assessment of insecticidal lipids prepared from Beauveria bassiana K4B3 at different timepoints.

### Methods

Cultures of Beauveria bassiana K4B3 were grown in shaken flasks and in a biofermentor as described above. 16 g samples were extracted at each 24 hour timepoint over 10 days, and lipids were extracted with methanol as described above. All samples were freeze dried, before being reconstituted at full strength (FS) in 1.28 ml H₂O and at a 1:5 dilution (1:5) by diluting 0.2 ml FS in 0.8 ml H₂O prior to assessment in the bioassays as described in Examples 1 and 2 above. Briefly, 300 ul of each sample was sprayed onto 40 aphids. Four negative controls (H₂O only) were also included in the assay.

### Results

Table 4 below presents data showing aphid mortality among the various treatment groups.

TABLE 4
Bioassay of aphid mortality

| Observation at 24th hour | |
|---|---|
| Replicate 1 | Replicate 2 |

| | | | Rep 1, | | | Rep 2, | Average |
|---|---|---|---|---|---|---|---|
| Treatments | Dead | Alive | % | Dead | Alive | % | (%) |
| D1 FS | 23 | 19 | 55 | 29 | 21 | 58 | 56 |
| D1 1:5 | 8 | 27 | 23 | 5 | 43 | 10 | 17 |
| D2 FS | 40 | 5 | 89 | 40 | 0 | 100 | 94 |
| D2 1:5 | 0 | 32 | 0 | 5 | 40 | 11 | 6 |
| D3 FS | 40 | 0 | 100 | 40 | 0 | 100 | 100 |
| D3 1:5 | 11 | 35 | 24 | 13 | 36 | 27 | 25 |
| D4 FS | 40 | 0 | 100 | 40 | 0 | 100 | 100 |
| D4 1:5 | 16 | 16 | 50 | 27 | 8 | 77 | 64 |
| D5 FS | 40 | 0 | 100 | 40 | 2 | 95 | 98 |
| D5 1:5 | 14 | 31 | 31 | 24 | 19 | 56 | 43 |
| D6 FS | 40 | 0 | 100 | 40 | 0 | 100 | 100 |
| D6 1:5 | 6 | 17 | 26 | 42 | 24 | 64 | 45 |
| D7 FS | 40 | 0 | 100 | 40 | 0 | 100 | 100 |
| D7 1:5 | 3 | 22 | 12 | 5 | 19 | 21 | 16 |
| D8 FS | 40 | 0 | 100 | 40 | 0 | 100 | 100 |
| D8 1:5 | 6 | 17 | 26 | 19 | 21 | 48 | 37 |
| D9 FS | 40 | 0 | 100 | 40 | 0 | 100 | 100 |
| D9 1:5 | 5 | 28 | 15 | 0 | 40 | 0 | 8 |
| D10 FS | 47 | 2 | 96 | 40 | 2 | 95 | 96 |
| D10 1:5 | 4 | 40 | 9 | 0 | 40 | 0 | 5 |
| H2O | 2 | 40 | 5 | 0 | 40 | 0 | 2 |
| H2O | 0 | 40 | 0 | 0 | 40 | 0 | 0 |

### Results

As can be seen in Table 4, all full strength samples (i.e., across each of the 10 days) killed aphids. Aphids killed by samples from days 1 to 5 were highly dessicated, whereas aphids killed by samples from days 5 to 10 were less desiccated.

### Discussion

This example indicates that insecticidal lipids are produced rapidly during growth, and production of the insecticidal activities is maintained over prolonged periods. Again, extracted lipids have potent insecticidal activity, typically killing 100% of target insects at full strength and frequently killing approximately 50% of target insects when more dilute.

### Example 6

### Bioassay of Insecticidal Lipids

### Introduction

This example describes the assessment of insecticidal lipids prepared from Beauveria bassiana K4B3.

### Methods

Lipid fractions from Beauveria bassiana K4B3 were extracted with 50% methanol:50% CH₂Cl₂ as described above. All samples were freeze dried, before being reconstituted at full strength (FS) and at a 1:5 dilution (1:5) prior to assessment in the bioassay. Briefly, 20 ul of each sample was administered to mosquito (Culex pervigilans) larvae. A negative control (MeOH only) and a positive control (S11) were also included in the assay. Mortality was assessed at 23 hours and at 48 hours after exposure.

### Results

Table 5 below presents data showing mosquito mortality among the various treatment groups.

TABLE 5
Bioassay of mosquito larvae mortality

| Mortality | |
|---|---|
| 23rd Hour | 48th Hour |

| | | | 23rd hr | | | 48th hr |
|---|---|---|---|---|---|---|
| Treatment | Dead | Alive | (%) | Dead | Alive | (%) |
| S1 | 0 | 10 | 0 | 3 | 7 | 30 |
| S2 | 2 | 8 | 20 | 4 | 6 | 40 |
| S3 | 4 | 6 | 40 | 5 | 5 | 50 |
| S4 | 2 | 8 | 20 | 3 | 7 | 30 |
| S5 | 6 | 4 | 60 | 8 | 2 | 80 |
| S6 | 3 | 7 | 30 | 5 | 5 | 50 |
| S7 | 4 | 6 | 40 | 6 | 4 | 60 |
| S8 | 6 | 4 | 60 | 8 | 2 | 80 |
| S3 1:5 | 2 | 8 | 20 | 8 | 2 | 80 |
| S3 1:10 | 2 | 6 | 25 | 5 | 3 | 73 |
| S4 1:5 | 4 | 6 | 40 | 8 | 2 | 71 |
| S4 1:10 | 2 | 7 | 22 | 3 | 6 | 57 |
| S5 1:5 | 4 | 6 | 40 | 7 | 3 | 50 |
| S5 1:10 | 3 | 5 | 38 | 7 | 1 | 88 |
| MeOH | 3 | 10 | 23 | 4 | 9 | 44 |
| S11 | 6 | 1 | 86 | 6 | 1 | 80 |

### Results

As can be seen in Table 5, fractions S5 and S8 in particular exhibited significant killing of mosquito larvae, even when diluted.

### Discussion

This example indicates that insecticidal lipids of the invention have potent insecticidal activity, killing a wide range of target insect species.

### Example 7

### Toxicity of Beauveria bassiana K4B3 Lipid in a Mammalian Model

### Introduction

This example describes an assessment of the toxicity of the K4B3 lipid in a mammalian model.

### Methods

K4B3 lipids are isolated as described above in Example 1.

Testing is conducted in mice according to OECD Guideline 425 (Acute Oral Toxicity-Up-and-down Procedure). Since this material is not expected to be highly toxic, the Limit Test with a single dose level of 2,000 mg/kg by oral intubation is chosen. This dose is the highest recommended by the OECD for evaluation of acute toxicity, except under exceptional circumstances.

A single 2,000 mg/kg dose of K4B3 lipid is administered by oral intubation to five female Swiss mice, as follows.

Test Conditions

Food is withdrawn from one of the mice at approximately 4 pm and its body weight is measured. Next morning, the mouse is weighed again and the weight of K4B3 lipid required to provide a dose of 2,000 mg/kg is calculated. This amount is weighed, and diluted with 150 μl of water. The whole volume is administered to the mouse by gavage.

After dosing, the mouse is allowed immediate access to food. It is observed intensively for 60 minutes after dosing and then at several intervals throughout the day of dosing and subsequent days, as specified in the OECD Guideline for the Testing of Chemicals, Revised Draft Guideline 425, October 2000. A second mouse is dosed with K4B4 polypeptide 48 hours after the first, again at a dose of 2,000 mg/kg body weight. The third, fourth, and fifth mice are subsequently dosed at 48 hour intervals, all at 2,000 mg/kg.

The mice are housed individually with water and food ad lib (except for the overnight fast before dosing). Mice are observed daily and body weight measured for 2 weeks following administration. Body weights are recorded 1 day, 1 week, and 2 weeks after dosing, after which the animals are killed by carbon dioxide inhalation and subjected to post-mortem examination.

### Results

Results showing no toxic effects after administration of the K4B3 complex, with mice remaining in good health throughout the observation period, feeding shortly after dosing, and behaving normally during the day of dosing and throughout the experiment, are indicative of no toxicity.

Body Weights.

Results showing unchanged mean body weights of the mice at various time intervals throughout the experiment are indicative of no toxicity.

After an overnight fast, mice typically lose an average of 2.4 grams in body weight. In circumstances where this loss is largely regained by the next day after access to food is restored following dosing, and in circumstances where the mice maintain their weight throughout the two-week observation period after dosing, no toxicity is indicated.

Post-Mortem Findings.

Results showing that no abnormalities are observed in the mice at necropsy, and that the weights of the livers, kidneys, spleens, hearts, lungs and intestine (pylorus to anus) of the mice are within their normal range, are indicative of no toxicity.

### Discussion

Results showing oral administration of K4B3 lipid to mice at a dose of 2,000 mg/kg causes no discernable adverse effects, wherein no deaths occur, no abnormalities are noted at necropsy, organ weights are within the normal range, and the behavior of the mice is entirely normal are indicative of low acute toxicity.

Such results are indicative that K4B3 lipid exhibits low acute oral toxicity, with an LD₅₀ greater than 2,000 mg/kg body weight. Such a result indicates that the K4B3 lipid would be classified in the lowest hazard category under the New Zealand Hazardous Substances and New Organisms (HSNO) Act 1996.

### INDUSTRIAL APPLICATION

As will be evident from the above description, the present invention provides insecticidal lipids from filamentous fungi together with compositions comprising said lipids useful for the control of insects, such as phytopathogenic insects. The use of such lipids and lipid fractions in methods to control insects, such as phytopathogenic insects, are also provided. The lipids and compositions of the invention have utility in a wide range of agricultural and horticultural applications.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art.

### PUBLICATIONS

Anis, A. I. M.; Brennan, P. 1982 Susceptibility of different populations of glasshouse whitefly Trialeurodes vaporariorum, Westwood to a range of chemical insecticides. Faculty of General Agriculture University College of Dublin, Research report 1980-1981: 55.
Elhag, E. A.; Horn, D. J. 1983 Resistance of greenhouse whitefly (Homoptera: Aleyrodidae) to insecticides in selected Ohio greenhouses. Journal of Economic Entomology 76: 945-948.
Georghiou, G. P. 1981 The occurrence of resistance to pesticides in arthropods, an index of cases reported through 1980. FAO of UN, Rome 1981. 172 p.
Gorman, K.; Devine, G. J.; Denholm, 1. 2000 Status of pesticide resistance in UK populations of glasshouse whitefly, Trialeurodes vaporariorum, and the two-spotted spider mite, Tetranychus urticae. The BCPC Conference: Pests and diseases: 1: 459-464
Grossman, J. 1994 Onion thrips. IPM Practitioner. 16(4): 12-13
Hommes, M. 1986 Insecticide resistance in greenhouse whitefly (Trialeurodes vaporariorum, Westw.) to synthetic pyrethroids. Mitteilungen aus der Biologischen Bundesanstalt fur Land-und Forstwirtschaft 232: 376.
OECD 1998: Guidelines for the Testing of Chemicals. www.oecd.org
Wardlow, L. R. 1985 Pyrethroid resistance in glasshouse whitefly (Trialeurodes vaporariorum, Westw.). Mededelingen van de Faculteit Landbouwwetenschappen, Rijksuniversiteit, Gent 50 (2b): 164-165.

1.-84. (canceled)

85. An isolated, purified or substantially pure lipid of formula I 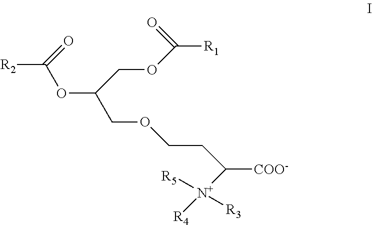
wherein R₁ and R₂, which can be the same or different, are each the aliphatic portion of a fatty acid, and wherein R3, R4 and R5 are each independently selected from the group comprising or consisting of H, hydroxyl, carboxyl, amide, unsubstituted or substituted alkyl, unsubstituted or substituted alkenyl, unsubstituted or substituted alkynyl, and unsubstituted or substituted aryl moieties.

86. The lipid according to claim 85 wherein R₁ and R₂ are independently selected from the group comprising the aliphatic portion of C16 saturated fatty acids, C18 saturated fatty acids, C18 mono-unsaturated fatty acids, C18 di-unsaturated fatty acids and C18 tri-unsaturated fatty acids.

87. The lipid according to claim 85 wherein R1 or R2 or both R1 and R2 is the aliphatic portion of a C16 saturated fatty acid.

88. The lipid according to claim 85 wherein R1 or R2 or both R1 and R2 is the aliphatic portion of a C18 saturated fatty acid.

89. The lipid according to claim 85 wherein R1 or R2 or both R1 and R2 is the aliphatic portion of a C18 mono-unsaturated fatty acid.

90. The lipid according to claim 85 wherein R1 or R2 or both R1 and R2 is the aliphatic portion of a C18 di-unsaturated fatty acid.

91. The lipid according to claim 85 wherein R1 or R2 or both R1 and R2 is the aliphatic portion of a C18 tri-unsaturated fatty acid.

92. The lipid according to claim 85 wherein
(i) R1 is the aliphatic portion of a C16 saturated fatty acid, or
(ii) R1 is the aliphatic portion of a C16 saturated fatty acid and R2 is a C18 mono-unsaturated fatty acid, or
(iii) R1 is the aliphatic portion of a C16 saturated fatty acid and R2 is a C18 di-unsaturated fatty acid, or
(iv) R2 is the aliphatic portion of a C16 saturated fatty acid, or
(v) R2 is the aliphatic portion of a C16 saturated fatty acid and R1 is the aliphatic portion of a C18 mono-unsaturated fatty acid, or
(vi), R2 is the aliphatic portion of a C16 saturated fatty acid and R1 is the aliphatic portion of a C18 di-unsaturated fatty acid, or
(vii) R2 is the aliphatic portion of a C16 saturated fatty acid and R1 is the aliphatic portion of a C18 di-unsaturated fatty acid, or
(viii) R1 is the aliphatic portion of a C18 saturated fatty acid, or
(ix) R1 is the aliphatic portion of a C18 saturated fatty acid and R2 is the aliphatic portion of a C18 mono-unsaturated fatty acid, or
(x) R1 is the aliphatic portion of a C18 saturated fatty acid and R2 is the aliphatic portion of a C18 di-unsaturated fatty acid, or
(xi) R1 is the aliphatic portion of a C18 saturated fatty acid and R2 is the aliphatic portion of a C18 tri-unsaturated fatty acid, or
(xii) R2 is the aliphatic portion of a C18 saturated fatty acid, or
(xiii) R2 is the aliphatic portion of a C18 saturated fatty acid and R1 is the aliphatic portion of a C18 mono-unsaturated fatty acid, or
(xiv) R2 is the aliphatic portion of a C18 saturated fatty acid and R1 is the aliphatic portion of a C18 di-unsaturated fatty acid, or
(xv) R2 is the aliphatic portion of a C18 saturated fatty acid and R1 is the aliphatic portion of a C18 tr-unsaturated fatty acid, or
(xvi) R1 is the aliphatic portion of a C18 mono-unsaturated fatty acid, or
(xvii) R1 is the aliphatic portion of a C18 mono-unsaturated fatty acid and R2 is the aliphatic portion of a C18 mono-unsaturated fatty acid, or
(xviii) R1 is the aliphatic portion of a C18 mono-unsaturated fatty acid and R2 is the aliphatic portion of a C18 di-unsaturated fatty acid, or
(xix) R1 is the aliphatic portion of a C18 mono-unsaturated fatty acid and R2 is the aliphatic portion of a C18 tri-unsaturated fatty acid, or
(xx) R2 is the aliphatic portion of a C18 mono-unsaturated fatty acid, or
(xxi) R2 is the aliphatic portion of a C18 mono-unsaturated fatty acid and R1 is the aliphatic portion of a C18 mono-unsaturated fatty acid, or
(xxii) R2 is the aliphatic portion of a C18 mono-unsaturated fatty acid and R1 is the aliphatic portion of a C18 di-unsaturated fatty acid, or
(xxiii) R2 is the aliphatic portion of a C18 mono-unsaturated fatty acid and R1 is the aliphatic portion of a C18 tri-unsaturated fatty acid, or
(xxiv) R1 is the aliphatic portion of a C18 di-unsaturated fatty acid, or
(xxv) R1 is the aliphatic portion of a C18 di-unsaturated fatty acid and R2 is the aliphatic portion of a C18 di-unsaturated fatty acid, or
(xxvi) R1 is the aliphatic portion of a C18 di-unsaturated fatty acid and R2 is the aliphatic portion of a C18 tri-unsaturated fatty acid, or
(xxvii) R2 is the aliphatic portion of a C18 di-unsaturated fatty acid, or
(xxviii) R2 is the aliphatic portion of a C18 di-unsaturated fatty acid and R1 is the aliphatic portion of a C18 tri-unsaturated fatty acid, or
(xxix) R1 is the aliphatic portion of a C18 tri-unsaturated fatty acid, or
(xxx) R1 is the aliphatic portion of a C18 tri-unsaturated fatty acid and R2 is the aliphatic portion of a C18 tri-unsaturated fatty acid, or
(xxxi) R2 is the aliphatic portion of a C18 tri-unsaturated fatty acid.

93. The lipid according to claim 85 wherein R3, R4, R5 are each H or methyl.

94. The lipid according to claim 85 wherein the lipid is 1,2-diacylglyceryl-3-O-4′-(N,N,N-trimethyl)-homoserine (diacylglyceryl-N,N,N-trimethylhomoserine, DGTS).

95. The lipid according to claim 85 wherein the lipid is 1,2-diacylglyceryl-3-O-2′-(hydroxymethyl)-(N,N,N-trimethyl)-β-alanine, or 1,2-diacylglyceryl-3-O-carboxy-(hydroxymethyl)-choline.

96. A composition comprising one or more lipids according to claim 85 in an insecticidally-effective amount, together with one or more carriers.

97. The composition of claim 96, wherein the composition is or comprises a cell extract, cell suspension, cell homogenate, cell lysate, cell supernatant, cell filtrate, cell pellet, or one or more cells of or from a culture of one or more fungi of the phylum Ascomycota.

98. The composition according to claim 96 enriched in or to which has been added one or more lipids.

99. The composition according to claims 96 wherein the composition comprises one or more fungi in a reproductively viable form and amount.

100. The composition according to claim 96 wherein the fungi of the phylum Ascomycota is Beauveria bassiana strain K4B3 (NMIA No. V08/025855 deposited 14 Oct. 2008), or wherein the composition comprises in a reproductively viable form and amount one or more strains selected from Beauveria bassiana K4B3 NMIA No. V08/025855 or a strain having the identifying characteristics thereof; Lecanicillium muscarium strain K4V1 (NMIA No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

101. A method of controlling one or more insects, the method comprising contacting the one or more insects with or applying to a locus an effective amount of one or more lipids according to claim 85.

102. A method of reversing, wholly or in part, the resistance of an insect to one or more insecticides or one or more entomopathogenic agents, the method comprising applying to a locus, or contacting the insect with, one or more lipids according to claim 85 or a functional variant thereof.

103. The method of claim 102 comprising applying to a locus, or contacting the insect with, the one or more lipids together with one or more insecticides or one or more entomopathogenic agents, or any combination thereof, wherein the one or more insecticides or one or more entomopathogenic agents administered is the same as that to which the insect is or is predicted to be or become resistant.

104. The method according to claim 102 wherein when present the one or more fungi is selected from Beauveria bassiana K4B3 NMIA No. V08/025855 or a strain having the identifying characteristics thereof; Lecanicillium muscarium strain K4V1 (NMIA No. NM05/44593) or a strain having the identifying characteristics thereof; Lecanicillium muscarium strain K4V2 (NMIA Accession No. NM05/44594) or a strain having the identifying characteristics thereof; Lecanicillium muscarium strain K4V4 (NMIA Accession No. NM06/00007) or a strain having the identifying characteristics thereof; Beauveria bassiana strain K4B1 (NMIA Accession No. NM05/44595) or a strain having the identifying characteristics thereof; Beauveria bassiana strain K4B2 (NMIA Accession No. NM06/00010) or a strain having the identifying characteristics thereof; Lecanicillium longisporum strain KT4L1 (NMIA Accession No. NM06/00009) or a strain having the identifying characteristics thereof; and Paecilomyces fumosoroseus strain K4P1 (NMIA Accession No. NM06/00008) or a strain having the identifying characteristics thereof.

105. A method for producing a biological control composition, the method comprising:
providing a culture of one or more fungi of the phylum Ascomycota, maintaining the culture under conditions suitable for production of at least one lipid according to claim 85; and
i) combining the at least one lipid with a carrier, or
ii) combining the at least one lipid with one or more entomopathogenic fungi described herein, or
iii) separating the at least one lipid from the fungi or fungal culture, or
iv) at least partially purifying or isolating the at least one lipid, or
v) any combination of two or more of (i) to (iv),
thereby to form the composition.

106. A method of preparing an insecticidal lipid, the method comprising
providing an organic solvent extraction of a culture or culture extract of one or more fungi of the phylum Ascomycota,
at least partially separating one or more lipids according to claim 85 from one or more other compounds, and
recovering the one or more lipids.

107. The method according to claim 108 wherein the organic solvent is or comprises chloroform, dichloromethane, an alkanol including a short chain alkyl alcohol, such as but not limited to methanol, ethanol, propanol, iso-propanol, butanol, or any combination of two or more thereof.

108. The method according to claim 105 wherein the culture is a culture of Beauveria bassiana K4B3 NMIA No. V08/025855 or a strain having the identifying characteristics thereof.
